(19) United States
(12) Patent Application Publication
(10) Pub. No.: US 2003/0013041 A1
(43) Pub. Date: Jan. 16, 2003

(54) Optical data carrier comprising a cationic aminoheterocyclic dye as light-absorbent compound in the information layer

(76) Inventors: Horst Berneth, Leverkusen (DE); Friedrich-Karl Bruder, Krefeld (DE); Wilfried Haese, Odenthal (DE); Rainer Hagen, Leverkusen (DE); Karin Hassenruck, Dusseldorf (DE); Serguei Kostromine, Swisttal (DE); Peter Landenberger, Koln (DE); Rafael Oser, Krefeld (DE); Thomas Sommermann, Gladbach (DE); Josef-Walter Stawitz, Odenthal (DE); Thomas Bieringer, Odenthal (DE)

Correspondence Address: BAYER CORPORATION, PATENT DEPARTMENT, 100 BAYER ROAD, PITTSBURGH, PA, 15205, US

(21) Appl. No.: 10/102,136

(22) Filed: Mar. 20, 2002

(30) Foreign Application Priority Data
Mar. 28, 2001 (DE) 10115227.2
Apr. 6, 2001 (DE) 10117462.4
Jul. 25, 2001 (DE) 10136063.0
Jul. 25, 2001 (DE) 10136064.9

(51) Int. Cl. G11B 7/24; C07D 317/00; C07D 327/04; C07D 339/02

(52) U.S. Cl. 430/270.18; 430/270.15; 430/945; 428/64.8; 549/31; 549/430; 549/464

(57) ABSTRACT

Optical data carrier comprising a preferably transparent substrate which may, if desired, have previously been coated with one or more reflection layers and to whose surface a light-writeable information layer, if desired one or more reflection layers and if desired a protective layer or a further substrate or a covering layer have been applied, which can be written on or read by means of blue, red or infrared light, preferably laser light, where the information layer comprises a light-absorbent compound and, if desired, a binder, characterized in that at least one cationic aminoheterocyclic dye is used as light-absorbent compound.

The invention relates to a write-once optical data carrier comprising a cationic aminoheterocyclic dye as light-absorbent compound in the information layer, to a process for its production and also to the application of the abovementioned dyes to a polymer substrate, in particular polycarbonate, by spin coating or vapour deposition.

Write-once optical data carriers using specific light-absorbent substances or mixtures thereof are particularly suitable for use in high-density writeable optical data stores which operate with blue laser diodes, in particular GaN or SHG laser diodes (360-460 nm) and/or for use in DVD-R or CD-R disks which operate with red (635-660 nm) or infrared (780-830 nm) laser diodes.

The write-once compact disk (CD-R, 780 nm) has recently experienced enormous volume growth and represents the technically established system.

The next generation of optical data stores—DVDs—is currently being introduced onto the market. Through the use of shorter-wave laser radiation (635-660 nm) and higher numerical aperture NA, the storage density can be increased. The writeable format in this case is DVD-R.

Today, optical data storage formats which use blue laser diodes (based on GaN, JP-A 08 191 171 or Second Harmonic Generation SHG JP-A-09 050 629) (360 nm -460 nm) with high laser power are being developed. Writeable optical data stores will therefore also be used in this generation. The achievable storage density depends on the focusing of the laser spot on the information plane. Spot size scales with the laser wavelength λ/NA. NA is the numerical aperture of the objective lens used. In order to obtain the highest possible storage density, the use of the smallest possible wavelength λ is the aim. At present 390 nm is possible on the basis of semiconductor laser diodes.

The patent literature describes dye-based writeable optical data stores which are equally suitable for CD-R and DVD-R systems (JP-A 11 043 481 and JP-A 10 181 206). To achieve a high reflectivity and a high modulation height of the read-out signal and also to achieve sufficient sensitivity in writing, use is made of the fact that the IR wavelength of 780 nm of CD-Rs is located at the foot of the long wavelength flank of the absorption peak of the dye and the red wavelength of 635 nm or 650 nm of DVD-Rs is located at the foot of the short wavelength flank of the absorption peak of the dye. In JP-A 02 557 335, JP-A 10 058 828, JP-A 06 336 086, JP-A 02 865 955, WO-A 09 917 284 and U.S. Pat. No. 5,266,699, this concept is extended to the 450 nm working wavelength region on the short wavelength flank and the red and IR region on the long wavelength flank of the absorption peak.

Apart from the abovementioned optical properties, the writeable information layer comprising light-absorbent organic substances has to have a substantially amorphous morphology to keep the noise signal during writing or reading as small as possible. For this reason, it is particularly preferred that crystallization of the light-absorbent substances be prevented in the application of the substances by spin coating from a solution, by vapour deposition and/or sublimation during subsequent covering with metallic or dielectric layers under reduced pressure.

The amorphous layer comprising light-absorbent substances preferably has a high heat distortion resistance, since otherwise further layers of organic or inorganic material which are applied to the light-absorbent information layer by sputtering or vapour deposition would form blurred boundaries due to diffusion and thus adversely affect the reflectivity. Furthermore, a light-absorbent substance which has insufficient heat distortion resistance can, at the boundary to a polymeric support, diffuse into the latter and once again adversely affect the reflectivity.

A light-absorbent substance whose vapour pressure is too high can sublime during the abovementioned deposition of further layers by sputtering or vapour deposition in a high vacuum and thus reduce the layer thickness to below the desired value. This in turn has an adverse effect on the reflectivity.

It is therefore an object of the invention to provide suitable compounds which satisfy the high requirements (e.g. light stability, favourable signal/noise ratio, damage-free application to the substrate material, and the like) for use in the information layer in a write-once optical data carrier, in particular for high-density writeable optical data store formats in a laser wavelength range from 340 to 830 nm.

Surprisingly, it has been found that light-absorbent compounds selected from the group of cationic aminoheterocyclic dyes can satisfy the abovementioned requirement profile particularly well.

The invention accordingly provides an optical data carrier comprising a preferably transparent substrate which may, if desired, have previously been coated with one or more reflection layers and to whose surface a light-writeable information layer, if desired one or more reflection layers and if desired a protective layer or a further substrate or a covering layer have been applied, which can be written on or read by means of blue, red or infrared light, preferably laser light, where the information layer comprises a light-absorbent compound and, if desired, a binder, characterized in that at least one cationic aminoheterocyclic dye is used as light-absorbent compound.

The light-absorbent compound should preferably be able to be changed thermally. The thermal change preferably occurs at a temperature of <600° C., particularly preferably at a temperature of <400° C., very particularly preferably at a temperature of <300° C., in particular <200° C. Such a change can be, for example, a decomposition or chemical change of the chromophoric centre of the light-absorbent compound.

Preference is given to a cationic aminoheterocyclic dye of the formula I

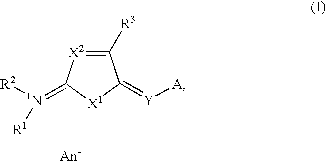

where

X¹ represents O or S,

X² represents CR¹⁰ or N,

R³ represents hydrogen, C₁-C₆-alkyl, halogen, hydroxy, C₆-C₁₀-aryl, NR⁸R⁹ or —CH═Y—A,

R¹, R², R⁸ and R⁹ represent, independently of one another, hydrogen, C₁-C₆-alkyl, C₅-C₇-cycloalkyl, C₆-C₁₀-aryl or C₇-C₁₅-aralkyl or

NR¹R² and NR⁸R⁹ represent, independently of one another, pyrrolidino, morpholino, piperazino or piperidino,

R¹⁰ represents hydrogen, cyano, C₁-C₆-alkyl, halogen or C₆-C₁₀-aryl,

Y represents ═Y¹—(Y²═Y³)ₙ—,

Y¹ to Y³ represent, independently of one another, N or C—R¹⁸,

n represents 0 or 1,

R¹⁸ represents hydrogen, cyano or C₁-C₃-alkyl, where, if n≅1, the radicals R¹⁸ of Y¹; Y², Y¹; Y³ or Y²; Y³ may in each case form a bridge,

A represents a radical of the formula

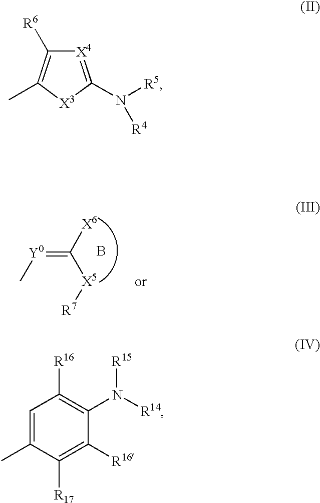

where

x³ represents O or S,

X⁴ represents CR¹¹ or N,

R⁶ represents hydrogen, C₁-C₆-alkyl, halogen, hydroxy, C₆-C₁₀-aryl or NR¹²R¹³ or

R³ and R⁶ may form an —O—, —CH₂— or —C(CH₃)₂— bridge or a bridge of the formula

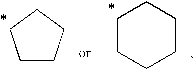

where two single bonds go out from the asterisked (*) carbon atom,

R⁴, R⁵, R¹² and R¹³ represent, independently of one another, hydrogen, C₁-C₆-alkyl, C₅-C₇-cycloalkyl, C₆-C₁₀-aryl or C₇-C₁₅-aralkyl or

NR⁴R⁵ and NR¹²R¹³ represent, independently of one another, pyrrolidino, morpholino, piperazino or piperidino,

R¹¹ represents hydrogen, cyano, C₁-C₆-alkyl, halogen or C₆-C₁₀-aryl,

X⁵ represents nitrogen or

X⁵—R⁷ represents S,

X⁶ represents O, S, N—R¹⁹,CR²⁰ or CR²⁰R²¹,

Y⁰ represents N or C—R¹⁸,

R⁷ and R¹⁹ represent, independently of one another, C₁-C₁₆-alkyl, C₃-C₆-alkenyl, C₅-C₇-cycloalkyl or C₇-C₁₆-aralkyl,

R²⁰ and R²¹ represent, independently of one another, C₁-C₄-alkyl or

CR²⁰R²¹ represents a bivalent radical of the formula

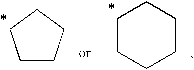

where the two bonds go out from the asterisked (*) ring atom,

B together with X⁵, X⁶ and the carbon atom between them represents a five- or six-membered aromatic or pseudoaromatic or partially hydrogenated hetero-cyclic ring which can contain from 1 to 4 heteroatoms and/or may be benzo- or naphtho-fused and/or substituted by nonionic radicals,

R¹⁴ and R¹⁵ represent, independently of one another, hydrogen, C₁-C₁₆-alkyl, C₄-C₇-cycloalkyl, C₇-C₁₆-aralkyl, C₆-C₁₀-aryl or a heterocyclic radical or

NR¹⁴R¹⁵ represents a five- or six-membered saturated ring which is bound via N and may additionally contain an N or O atom and/or be substituted by nonionic radicals,

R¹⁶ and R^{16′} represent, independently of one another, hydrogen, C₁-C₁₆-alkyl, C₁-C₁₆-alkoxy or halogen or

R¹⁶; R¹⁴ and/or R¹⁶; R¹⁵ form a two- or three-membered bridge which may contain an O or N atom and/or be substituted by nonionic radicals,

R¹⁷ represents hydrogen, C₁-C₁₆-alkyl, C₁-C₁₆-alkoxy, halogen, cyano, C₁-C₄-alkoxycarbonyl, O—CO—R²², NR²³—CO—R²², O—SO₂—R²² or NR²³—SO₂—R²²,

R²² represents hydrogen, C₁-C₁₆-alkyl, C₄-C₇-cycloalkyl, C₇-C₁₆-aralkyl, C₁-C₁₆-alkoxy, mono- or bis-C₁-C₁₆-alkylamino, C₆-C₁₀-aryl, C₆-C₁₀-aryloxy, C₆-C₁₀₋-arylamino or a heterocyclic radical,

R²³ represents hydrogen or C₁-C₄-alkyl,

and

An⁻ represents an anion.

Preference is likewise given to a cationic aminoheterocyclic dye of the formula I

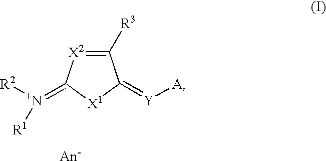

where

X¹ represents O, S or Se,

X² represents CR¹⁰ or N,

R³ represents hydrogen, C₁-C₆-alkyl, halogen, hydroxy, C₆-C₁₀-aryl, NR⁸R⁹ or —CH═Y—A,

R¹, R², R⁸ and R⁹ represent, independently of one another, hydrogen, C₁-C₆-alkyl, C₅-C₇-cycloalkyl, C₆-C₁₀-aryl or C₇-C₁₅-aralkyl or

NR¹R² and NR⁸R⁹ represent, independently of one another, pyrrolidino, morpholino, piperazino or piperidino,

R¹⁰ represents hydrogen, cyano, C₁-C₆-alkyl, halogen or C₆-C₁₀-aryl,

Y represents ═Y¹—(Y²═Y³)ₙ—,

Y¹ to Y³ represent, independently of one another, N or C—R¹⁸,

n represents 0 or 1,

R¹⁸ represents hydrogen, cyano, C₁-C₃-alkyl or a radical A, where, if n=1, the radicals R¹⁸ of Y¹; Y²; Y¹; Y³ or Y²; Y³ may in each case form a bridge,

A represents a radical of the formula

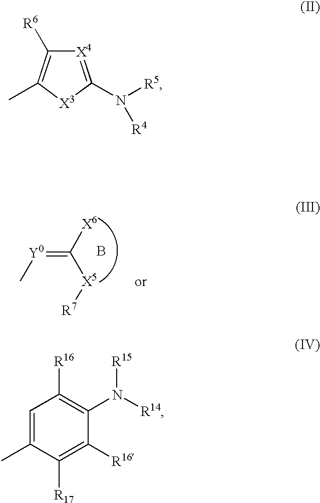

where

X³ represents O, S or Se,

X⁴ represents CR¹¹ or N,

R⁶ represents hydrogen, C₁-C₆-alkyl, halogen, hydroxy, C₆-C₁₀-aryl or NR¹²R¹³ or

R³ and R⁶ may form an —O—, —CH₂— or —C(CH₃)₂— bridge or a bridge of the formula

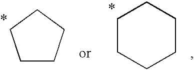

, where two single bonds go out from the asterisked (*) carbon atom,

R⁴, R⁵, R¹² and R¹³ represent, independently of one another, hydrogen, C₁-C₆-alkyl, C₅-C₇-cycloalkyl, C₆-C₁₀-aryl or C₇-C₁₅-aralkyl or

NR⁴R⁵ and NR¹²R¹³ represent, independently of one another, pyrrolidino, morpholino, piperazino or piperidino,

R¹¹ represents hydrogen, cyano, C₁-C₆-alkyl, halogen or C₆-C₁₀-aryl,

X⁵ represents nitrogen or

X⁵-R⁷ represents S,

X⁶ represents O, S, N—R¹⁹, CR²⁰ or CR²⁰R²¹,

Y⁰ represents N or C—R⁸,

R⁷ and R¹⁹ represent, independently of one another, C₁-C₁₆-alkyl, C₃-C₆-alkenyl, C₅-C₇-cycloalkyl or C₇-C₁₆-aralkyl,

R²⁰ and R²¹ represent, independently of one another, C₁-C₄-alkyl or

CR²⁰R²¹ represents a bivalent radical of the formula

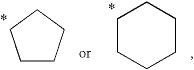

where the two bonds go out from the asterisked (*) ring atom,

B together with X⁵, X⁶ and the carbon atom between them represents a five- or six-membered aromatic or pseudoaromatic or partially hydrogenated hetero-cyclic ring which can contain from 1 to 4 heteroatoms and/or may be benzo- or naphtho-fused and/or substituted by nonionic radicals,

R¹⁴ and R¹⁵ represent, independently of one another, hydrogen, C₁-C₁₆-alkyl, C₄-C₇-cycloalkyl, C₇-C₁₆-aralkyl, C₆-C₁₀-aryl or a heterocyclic radical or

NR¹⁴R¹⁵ represents a five- or six-membered saturated ring which is bound via N and may additionally contain an N or O atom and/or be substituted by nonionic radicals,

R¹⁶ and R^{16′} represent, independently of one another, hydrogen, C₁-C₁₆-alkyl, C₁-C₁₆-alkoxy or halogen or

R¹⁶; R¹⁴ and/or R^{16′}; R¹⁵ form a two- or three-membered bridge which may contain an O or N atom and/or be substituted by nonionic radicals,

R¹⁷ represents hydrogen, C₁-C₁₆-alkyl, C₁-C₁₆-alkoxy, halogen, cyano, C₁-C₄-alkoxycarbonyl, O—CO—R²², NR²³—CO—R²², O—SO₂—R²² or NR²³—SO₂—R²²,

R²² represents hydrogen, C₁-C₁₆-alkyl, C₄-C₇-cycloalkyl, C₇-C₁₆-aralkyl, C₁-C₁₆-alkoxy, mono- or bis-C₁-C₁₆-alkylamino, C₆-C₁₀-aryl, C₆-C₁₀-aryloxy, C₆-C₁₀₋-arylamino or a heterocyclic radical,

R²³ represents hydrogen or C₁-C₄-alkyl, and

An⁻ represents an anion.

Possible nonionic radicals are, for example, C₁-C₄-alkyl, C₁-C₄-alkoxy, halogen, cyano, nitro, C₁-C₄-alkoxycarbonyl, C₁-C₄-alkylthio, C₁-C₄-alkanoylamino, benzoyl-amino, mono- or di-C₁-C₄-alkylamino.

Alkyl, alkoxy, aryl and heterocyclic radicals may, if desired, bear further radicals such as alkyl, halogen, nitro, cyano, CO—NH₂, alkoxy, trialkylsilyl, trialkylsiloxy or phenyl, the alkyl and alkoxy radicals may be straight-chain or branched, the alkyl radicals may be partially halogenated or perhalogenated, the alkyl and alkoxy radicals may be ethoxylated or propoxylated or silylated, adjacent alkyl and/or alkoxy radicals on aryl or heterocyclic radicals may together form a three- or four-membered bridge and the heterocyclic radicals may be benzo-fused and/or quatemized.

Preference is given to the ring B of the formula

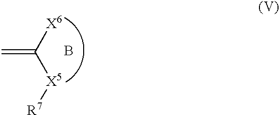

representing benzothiazol-2-ylidene, 1,3-thiazol-2-ylidene, isothiazol-3-ylidene, benzoxazol-2-ylidene, 1,3-oxazol-2-ylidene, isoxazol-3-ylidene, benzimidazol-2-ylidene, imidazol-2-ylidene, pyrazol-5-ylidene, 1,3,4-thiadiazol-2-ylidene, 1,3,4-oxadiazol-2-ylidene, 1,2,4-thiadiazol-5-ylidene, 1,3,4-triazol-2-ylidene, 1,3-dithiol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-yliden or pyrrolin-2-ylidene, where the rings mentioned may each be substituted by C₁-C₆-alkyl, C₁-C₆-alkoxy, fluorine, chlorine, bromine, iodine, cyano, nitro, C₁-C₆-alkoxy-carbonyl, C₁-C₆-alkylthio, C₁-C₆-acylamino, C₆-C₁₀-aryl, C₆-C₁₀-aryloxy, C₆-C₁₀-arylcarbonylamino, mono- or di-C₁-C₆-alkylamino, N—C₁C₆-alkyl-N—C₆-C₁₀-aryl-amino, pyrrolidino, morpholino or piperidino.

Preference is likewise given to the ring B of the formula

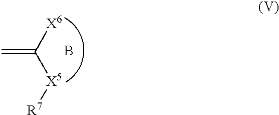

representing benzothiazol-2-ylidene, 1,3-thiazol-2-ylidene, isothiazol-3-ylidene, benzoxazol-2-ylidene, 1,3-oxazol-2-yliden isoxazol-3-ylidene, benzimidazol-2-ylidene, imidazol-2-ylidene, pyrazol-5-ylidene, 1,3,4-thiadiazol-2-ylidene, 1,3,4-oxadiazol-2-ylidene, 1,2,4-thiadiazol-5-ylidene, 1,3,4-triazol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-yliden or pyrrolin-2-ylidene, where X⁵ represents N and is substituted by R⁷, or the ring B representing 1,3-dithiol-2-ylidene, where X⁵-R⁷ represents S,

where the rings mentioned may each be substituted by C₁-C₆-alkyl, C₁-C₆-alkoxy, fluorine, chlorine, bromine, iodine, cyano, nitro, C₁-C₆-alkoxycarbonyl, C₁-C₆-alkylthio, C₁-C₆-acylamino, C₆-C₁₀-aryl, C₆-C₁₀-aryloxy, C₆-C₁₀-arylcarbonylamino, mono- or di-C₁-C₆-alkylamino, N—C₁-C₆-alkyl-N—C₆-C₁₀-arylamino, pyrrolidino, morpholino or piperidino.

In a particularly preferred embodiment, the cationic aminoheterocyclic dye used is one of the formula (VI),

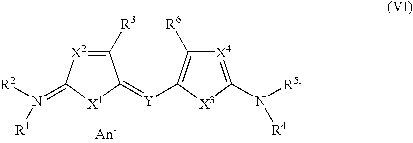

where

X¹ and X³ represent, independently of one another, O or S,

X² represents CR¹⁰ or N,

X⁴ represents CR¹¹ or N,

R³ represents hydrogen, methyl, ethyl, propyl, butyl, chlorine, phenyl, tolyl, methoxyphenyl, xylyl or NR⁸R⁹,

R⁶ represents hydrogen, methyl, ethyl, propyl, butyl, chlorine, phenyl, tolyl, methoxyphenyl, xylyl or NR¹²R¹³ or

R³ and R⁶ together form an —O—, —CH₂— or —C(CH₃)₂— bridge when n=0,

R¹, R², R⁴, R⁵, R⁸, R⁹, R¹² and R¹³ represent, independently of one another, hydrogen, methyl, ethyl, propyl, butyl, cyanoethyl, hydroxyethyl, hydroxy-propyl, cyclopentyl, cyclohexyl, phenyl, tolyl, methoxyphenyl, xylyl, benzyl, phenethyl or phenylpropyl or

NR¹R², NR⁴R⁵, NR⁸R⁹ and NR¹²R¹³ represent, independently of one another, pyrrolidino, morpholino, piperazino, N-methyl-piperazino or piperidino,

R¹⁰ and R¹¹ represent, independently of one another, hydrogen, cyano, methyl, ethyl, propyl, butyl, phenyl, tolyl, methoxyphenyl or xylyl,

Y represents ═Y¹—(Y²═Y³)ₙ— or N,

Y¹ to Y³ represent CH,

n represents 0 or 1 and

An⁻ represents an anion.

In a likewise preferred embodiment, the cationic arninoheterocyclic dyes used are ones of the formula (VI),

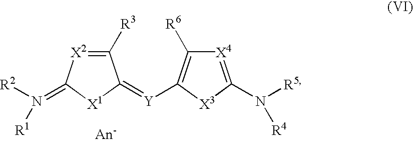

where

X¹, X³ and X³* represent, independently of one another, O, S or Se,

X² represents CR¹⁰ or N,

X⁴ and X⁴* represent, independently of one another, CR¹¹ or N,

R³ represents hydrogen, methyl, ethyl, propyl, butyl, chlorine, phenyl, tolyl, methoxyphenyl, xylyl or NR⁸R⁹,

R⁶ and R⁶* represent, independently of one another, hydrogen, methyl, ethyl, propyl, butyl, chlorine, phenyl, tolyl, methoxyphenyl, xylyl or N¹²R¹³ or

R³ and R⁶ together form an —O—, —CH₂— or —C(CH₃)₂— bridge when n=0,

R¹,R², R⁴, R⁴*, R⁵, R⁵*, R⁸, R⁹, R¹²,R¹³, R¹⁴* and R¹⁵* represent, independently of one another, hydrogen, methyl, ethyl, propyl, butyl, cyanoethyl, hydroxy-ethyl, hydroxypropyl, cyclopentyl, cyclohexyl, phenyl, tolyl, methoxyphenyl, xylyl, benzyl, phenethyl or phenylpropyl or

NR¹R², NR⁴R⁵, NR⁴*R⁵*, NR⁸R⁹, NR¹²R¹³ and NR¹⁴*R¹⁵* represent, independently of one another, pyrrolidino, morpholino, piperazino, N-methyl-piperazino or piperidino,

R¹⁰ and R¹¹ represent, independently of one another, hydrogen, cyano, methyl, ethyl, propyl, butyl, phenyl, tolyl, methoxyphenyl or xylyl,

Y represents ═Y¹—(Y²═Y³)ₙ— or N,

Y¹ to Y³ represent CH and Y¹ may also represent C—R¹⁸ when n=0,

R¹⁸ represents a radical of the formula

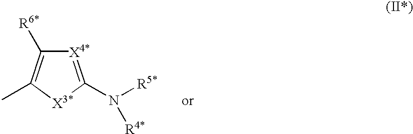

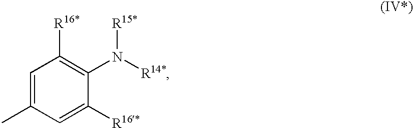

R¹⁶* and R^{16′}* represent, independently of one another, hydrogen, methyl, methoxy or chlorine or

R¹⁶*; R¹⁴* and/or R^{16′}*/R¹⁵* may form a —(CH₂)₂— or —(CH₂)₃— bridge,

n represents 0 or 1 and

An⁻ represents an anion.

Very particular preference is given to the members of the pairs X¹; X³, X²; X⁴, R¹; R⁴, R²; R⁵ and R³; R⁶ being identical in each case.

Very particular preference is likewise given to the dyes of the formula (VI),

in which

X¹ and X³ are identical and represent O or S,

X² and X⁴ are identical and represent CH or N,

R³ and R⁶ are identical and represent hydrogen, methyl, 2-propyl, tert-butyl, chlorine, phenyl or NR⁸R⁹,

R¹, R², R⁴, R⁵, R⁸ and R⁹ represent hydrogen, methyl, ethyl, propyl, butyl, cyano-ethyl, hydroxyethyl, hydroxypropyl, phenyl or benzyl, where the radicals of the groups R¹; R⁴; R⁸ and R²; R⁵; R⁹ are in each case identical, or

NR¹R², NR⁴R⁵ and NR⁸R⁹ are identical and represent pyrrolidino, morpholino or piperidino,

Y represents CH or ═CH—CH═CH—,

and

An⁻ represents an anion.

In a likewise particularly preferred embodiment, the cationic aminoheterocyclic dyes used are ones of the formula (VII),

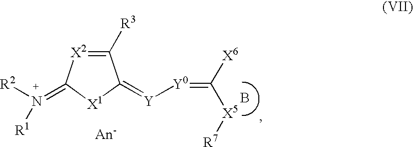

where

X¹ represents O or S,

X² represents CR¹⁰ or N,

R³ represents hydrogen, methyl, ethyl, propyl, butyl, chlorine, phenyl, tolyl, methoxyphenyl, xylyl or NR⁸R⁹,

R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or

NR¹R² and NR⁸R⁹ represent, independently of one another, pyrrolidino, morpholino, piperazino, N-methyl-piperazino or piperidino,

R¹⁰ represents hydrogen, cyano, methyl, ethyl, propyl, butyl, phenyl, tolyl, methoxyphenyl or xylyl,

the ring B together with X⁵ and X⁶ represents, when X⁵ represents N, benzothiazol-2-ylidene, 1,3-thiazol-2-ylidene, benzoxazol-2-ylidene, benzimidazol-2-ylidene, 3-H-indol-2-ylidene, dihydropyridin-2- or -4-ylidene, dihydroquinolin-2- or-4-ylidene, 1,3-thiazolin-2-ylidene, 1,3-oxazolin-2-ylidene, imidazolin-2-ylidene or pyrrolin-2-ylidene or, when X⁵-R⁷ represents S, represents 1,3-dithiol-2-ylidene, where the rings mentioned may each be substituted by up to three radicals selected from the group consisting of methyl, ethyl, trifluoromethyl, methoxy, ethoxy, fluorine, chlorine, bromine, cyano, nitro, methoxycarbonyl, ethoxycarbonyl, methylthio, formylamino, acetylamino, propionylamino, phenyl, tolyl, methoxyphenyl, phenoxy, benzoylamino, dimethylamino, diethylamino, dipropylamino, N-methyl-N-phenylamino, pyrrolidino, morpholino or piperidino,

R⁷ represents methyl, ethyl, propyl, butyl, cyanoethyl, hydroxyethyl, hydroxypropyl, allyl, cyclopentyl, cyclohexyl, benzyl, phenethyl or phenylpropyl,

Y represents ═Y¹—(Y²═Y³)ₙ— or N,

Y¹ to Y³ represent CH,

n represents 0 or 1,

Y⁰ represents CH or N and

An⁻ represents an anion.

In a likewise particularly preferred embodiment, the cationic aminoheterocyclic dyes used are ones of the formula (VII),

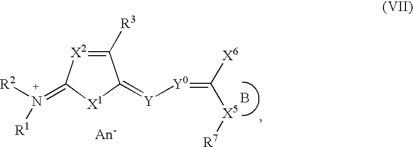

where

X¹ and X³* represent, independently of one another, O, S or Se,

X² represents CR¹⁰ or N,

X⁴* represents CR¹¹ or N,

R³ represents hydrogen, methyl, ethyl, propyl, butyl, chlorine, phenyl, tolyl, methoxyphenyl, xylyl or NR⁸R⁹,

R⁶* represents hydrogen, methyl, ethyl, propyl, butyl, chlorine, phenyl, tolyl, methoxyphenyl, xylyl or NR¹²R¹³,

R¹, R², R⁴*, R⁵*, R⁸, R⁹, R¹⁴* and R¹⁵* represent, independently of one another, hydrogen, methyl, ethyl, propyl, butyl, cyanoethyl, hydroxyethyl, hydroxy-propyl, cyclopentyl, cyclohexyl, phenyl, tolyl, methoxyphenyl, xylyl, benzyl, phenethyl or phenylpropyl or

NR¹R², NR⁴*R⁵*, NR⁸R⁹ and R¹⁴*R¹⁵* represent, independently of one another, pyrrolidino, morpholino, piperazino, N-methyl-piperazino or piperidino,

R¹⁰ and R¹¹ represent, independently of one another, hydrogen, cyano, methyl, ethyl, propyl, butyl, phenyl, tolyl, methoxyphenyl or xylyl,

the ring B together with X⁵, X⁶ and R⁷ represents, when X⁵ represents N, benzothiazol-2-ylidene, 1,3-thiazol-2-ylidene, benzoxazol-2-ylidene, benz-imidazol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-ylidene or pyrrolin-2-ylidene, where the heterocyclic radicals mentioned are each substituted on X⁵═N by R⁷, or, when X⁵—R⁷ represents S, represents 1,3-dithiol-2-ylidene, where the rings mentioned may each be substituted by up to three radicals selected from the group consisting of methyl, ethyl, trifluoromethyl, methoxy, ethoxy, fluorine, chlorine, bromine, cyano, nitro, methoxycarbonyl, ethoxycarbonyl, methylthio, formylamino, acetylamino, propionylamino, phenyl, tolyl, methoxyphenyl, phenoxy, benzoylamino, dimethylamino, diethylamino, dipropylamino, N-methyl-N-phenylamino, pyrrolidino, morpholino or piperidino,

R⁷ represents methyl, ethyl, propyl, butyl, cyanoethyl, hydroxyethyl, hydroxypropyl, allyl, cyclopentyl, cyclohexyl, benzyl, phenethyl or phenylpropyl,

Y represents ═Y¹—(Y²═Y³)ₙ— or N,

Y¹ to Y³ represent CH and Y¹ may also represent C—R¹⁸, when n=0,

R¹⁸ represents a radical of the formula

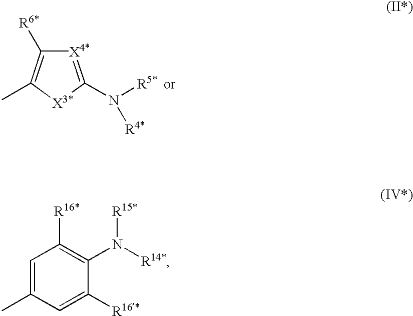

R¹⁶* and R^{16′}* represent, independently of one another, hydrogen, methyl, methoxy or chlorine or

R¹⁶* R¹⁴* and/or R^{16′}* /R¹⁵* may form a —(CH₂)₂— or —(CH₂)₃— bridge,

n represents 0 or 1,

Y⁰ represents CH or N and

An⁻ represents an anion.

Very particular preference is given to the dyes of the formula (VII)

in which

X¹ represents O or S,

X² represents CH or N,

R³ represents hydrogen, methyl, 2-propyl, tert-butyl, chlorine, phenyl or NR⁸R⁹,

R¹, R², R⁸ and R⁹ represent hydrogen, methyl, ethyl, propyl, butyl, cyanoethyl, hydroxyethyl, hydroxypropyl, phenyl or benzyl, where the radicals of the pairs R¹; R⁸ and R²; R⁹ are in each case identical, or

NR¹R² and NR⁸R⁹ are identical and represent pyrrolidino, morpholino or piperidino,

the ring B together with X⁵ and X⁶ represents, when X⁵ represents N, benzothiazol-2-ylidene, benzoxazol-2-ylidene, benzimidazol-2-ylidene, 3-H-indol-2-ylidene, dihydropyridin-2- or -4-ylidene, dihydroquinolin-2- or -4-ylidene, 1,3-thia-zolin-2-ylidene or pyrrolin-2-ylidene or, when X⁵—R⁷ represents S, represents 1,3-dithiol-2-ylidene, where the rings mentioned may each be substituted by up to two radicals selected from the group consisting of methyl, ethyl, trifluoromethyl, methoxy, ethoxy, fluorine, chlorine, bromine, cyano, nitro, methoxycarbonyl, ethoxycarbonyl, acetylamino, propionylamino, phenyl, phenoxy or benzoylamino,

R⁷ represents methyl, ethyl, propyl, butyl, cyanoethyl, hydroxyethyl, hydroxypropyl or benzyl,

Y and Y⁰ represent CH and

An⁻ represents an anion.

In a likewise particularly preferred embodiment, the cationic aminoheterocyclic dyes used are ones of the formula (VIII),

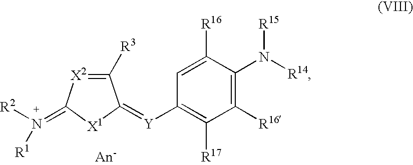

where

X¹ represents O or S,

X² represents CR¹⁰ or N,

R³ represents hydrogen, methyl, ethyl, propyl, butyl, chlorine, phenyl, tolyl, methoxyphenyl, xylyl or NR⁸R⁹,

R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or

NR¹R² and NR⁸R⁹ represent, independently of one another, pyrrolidino, morpholino, piperazino, N-methyl-piperazino or piperidino,

R¹⁰ represents hydrogen, cyano, methyl, ethyl, propyl, butyl, phenyl, tolyl, methoxyphenyl or xylyl,

R¹⁴ and R¹⁵ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl, phenylpropyl, pyridyl or a radical of the formula

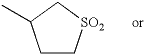

NR¹⁴R¹⁵ represents pyrrolidino, morpholino, piperazino, N-methyl-piperazino or piperidino,

R¹⁶ and R^{16′} represent, independently of one another, hydrogen, methyl, ethyl, methoxy, ethoxy or chlorine or

R¹⁶; R¹⁵ and/or R^{16′}; R¹⁴ form a —(CH₂)₂— or —(CH₂)₃— bridge which may be substituted by up to 3 methyl groups,

R¹⁷ represents hydrogen, methyl, ethyl, methoxy, ethoxy, chlorine, cyano, methoxycarbonyl, ethoxycarbonyl, O—CO—R²², NR²³—CO—R²², O—SO₂—R²² or NR²³—SO₂—R²²,

R²² represents methyl, ethyl, trifluoromethyl, cyclopentyl, cyclohexyl, benzyl, methoxy, ethoxy, dimethylamino, diethylamino, methylamino, phenyl, phenoxy, anilino or pyridyl,

R²³ represents hydrogen or methyl,

Y represents ═Y¹—(Y²═Y³)ₙ— or N,

Y¹ to Y³ represent CH,

n represents 0 or 1 and

An⁻ represents an anion.

In a likewise particularly preferred embodiment, the cationic aminoheterocyclic dyes used are ones of the formula (VIII),

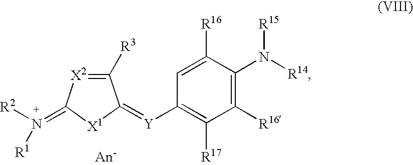

where

X¹ represents O, S or Se,

X² represents CR¹⁰ or N,

R³ represents hydrogen, methyl, ethyl, propyl, butyl, chlorine, phenyl, tolyl, methoxyphenyl, xylyl or NR⁸R⁹,

R¹, R², R⁸, R⁹, R¹⁴* and R¹⁵* represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or

NR¹R², NR⁸R⁹ and NR¹⁴*R¹⁵* represent, independently of one another, pyrrolidino, morpholino, piperazino, N-methyl-piperazino or piperidino,

R¹⁰ represents hydrogen, cyano, methyl, ethyl, propyl, butyl, phenyl, tolyl, methoxyphenyl or xylyl,

R¹⁴ and R¹⁵ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl, phenylpropyl, pyridyl or a radical of the formula

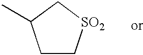

NR¹⁴R¹⁵ represents pyrrolidino, morpholino, piperazino, N-methyl-piperazino or piperidino,

R¹⁶ and R^{16′} represent, independently of one another, hydrogen, methyl, ethyl, methoxy, ethoxy or chlorine or

R¹⁶; R¹⁵ and/or R^{16′}; R¹⁴ form a —(CH₂)₂— or —(CH₂)₃— bridge which may be substituted by up to 3 methyl groups,

R¹⁷ represents hydrogen, methyl, ethyl, methoxy, ethoxy, chlorine, cyano, methoxycarbonyl, ethoxycarbonyl, O—CO—R²², NR²³—CO—R²², O—SO₂—R²² or NR²³—SO₂—R²²,

R²² represents methyl, ethyl, trifluoromethyl, cyclopentyl, cyclohexyl, benzyl, methoxy, ethoxy, dimethylamino, diethylamino, methylamino, phenyl, phenoxy, anilino or pyridyl,

R²³ represents hydrogen or methyl,

Y represents ═Y¹—(Y²═Y³)ₙ— or N,

Y¹ to Y³ represent CH and Y¹ may also represent C—R¹⁸ when n=0,

R¹⁸ represents a radical of the formula

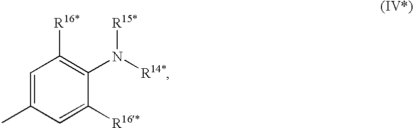

R¹⁶* and R^{16′}* represent, independently of one another, hydrogen, methyl, methoxy or chlorine or

R¹⁶*; R¹⁴* and/or R^{16′}*/R¹⁵* may form a —(CH₂)₂— or —(CH₂)₃— bridge,

n represents 0 or 1 and

An⁻ represents an anion.

Very particular preference is given to the dyes of the formula (VIII),

in which

X¹ represents O or S,

X² represents CH or N,

R³ represents hydrogen, methyl, 2-propyl, tert-butyl, chlorine, phenyl or NR⁸R⁹,

R¹, R², R⁸ and R⁹ represent hydrogen, methyl, ethyl, propyl, butyl, cyanoethyl, hydroxyethyl, hydroxypropyl, phenyl or benzyl, where the radicals of the pairs R¹; R⁸ and R²; R⁹ are in each case identical, or

NR¹R² and NR⁸N⁹ are identical and represent pyrrolidino, morpholino or piperidino,

R¹⁴ and R¹⁵ represent, independently of one another, hydrogen, methyl, ethyl, propyl, butyl, cyanoethyl, hydroxyethyl, hydroxypropyl, phenyl or benzyl or

NR¹⁴R¹⁵ represents pyrrolidino, morpholino or piperidino,

R¹⁶ represents hydrogen, methyl or methoxy or

R¹⁶; R¹⁵ form a —(CH₂)₂—, —(CH₂)₃— or —C(CH₃)₂—(CH₂)₂— bridge,

R^{16′} represents hydrogen,

R¹⁷ represents hydrogen, methyl, ethyl, methoxy, ethoxy, chlorine, cyano, methoxycarbonyl, ethoxycarbonyl, acetoxy, acetamino, benzoylamino, methanesulphonylamino or trifluoromethanesulphonylamino,

Y represents CH or N and

An⁻ represents an anion.

Possible anions An⁻ include all monovalent anions or one equivalent of a polyvalent or polymeric anion. Preference is given to colourless anions. Examples of suitable anions are chloride, bromide, iodide, tetrafluoroborate, perchlorate, hexafluoro-silicate, hexafluorophosphate, methosulphate, ethosulphate, C₁-C₁₀ -alkanesulphonate, C₁-C₁₀-perfluoroalkanesulphonate, unsubstituted or chloro-, hydroxy- or C₁-C₄-alkoxy-substituted C₁-C₁₀-alkanoate, unsubstituted or nitro-, cyano-, hydroxy-, C₁-C₂₅-alkyl-, perfluoro-C₁-C₄-alkyl-, C₁-C₄-alkoxycarbonyl- or chloro-substituted benzenesulphonate, or naphthalenesulphonate or biphenylsulphonate, unsubstituted or nitro-, cyano-, hydroxy-, C₁-C₄-alkyl-, C₁-C₄-alkoxy-, C₁-C₄-alkoxycarbonyl- or chloro-substituted benzenedisulphonate, naphthalenedisulphonate or biphenyldi-sulphonate, unsubstituted or nitro-, cyano-, C₁-C₄-alkyl-, C₁-C₄-alkoxy-, C₁-C₄-alkoxycarbonyl-, benzoyl-, chlorobenzoyl- or toluyl-substituted benzoate, the anion of naphthalenedicarboxylic acid, (diphenyl ether)disulphonate, tetraphenylborate, cyanotriphenylborate, tetra-C₁-C₂₀-alkoxyborate, tetraphenoxyborate, 7,8- or 7,9- dicarba-nido-undecaborate(1-) or (2-), which may, if desired, be substituted on the B and/or C atoms by one or two C₁-C₁₂-alkyl or phenyl groups, dodecahydro-dicarba-dodecaborate(2-) or B—C₁-C₁₂-alkyl-C-phenyl-dodecahydro-dicarbadodecaborate(1-), polystyrenesulphonate, polyacrylate, polymethacrylate, polymethallylsulphonate or polyaspartate.

Further suitable anions are the anions of dyes, preferably of dyes whose λₘₐₓ differs from the λₘₐₓ₂ or λₘₐₓ₃ of the dyes of the formula (I) by not more than 30 nm, preferably by not more than 20 nm, very particularly preferably by not more than 10 nm. Examples of such anionic dyes are oxonols, azo dyes, anthraquinone dyes or metal complex dyes such as formazanes or azo metal complex dyes.

Preference is given to bromide, iodide, tetrafluoroborate, perchlorate, hexafluoro-phosphate, methanesulphonate, trifluoromethanesulphonate, benzenesulphonate, toluenesulphonate, dodecylbenzenesulphonate, tetradecanesulphonate.

Mixtures of the cationic aminoheterocyclic dyes used according to the invention can also be employed, for example for optimizing the physical properties of the information layer.

It is likewise possible to use mixtures with other, preferably cationic dyes. The other dyes used in these mixtures are preferably ones whose λₘₐₓ differs from the λₘₐₓ₂ or λₘₐₓ₃ of the dyes of the formula (I) by not more than 30 nm, preferably by not more than 20 nm, very particularly preferably by not more than 10 nm. Examples which may be mentioned are dyes from the classes of cyanines, streptocyanines, hemicyanines, diazahemicyanines, nullmethines, enamine dyes, hydrazone dyes, di- or tri(het)arylmethane dyes, xanthene dyes, azine dyes (phenazines, oxazines, thiazines) or, for example, from the classes of azo dyes, anthraquinone dyes, neutrocyanines, porphyrins or phthalocyanines. Such dyes are known, for example, from H. Bemeth, Cationic Dyes in Ullmann's Encyclopedia of Industrial Chemistry, VCH, 6th edition.

In the case of a write-once optical data carrier according to the invention which is written on and read by means of the light of a blue laser, preference is also given to cationic aminoheterocyclic dyes whose absorption maximum λₘₐₓ₂ is in the range from 420 to 550 nm, where the wavelength λ_{½} at which the absorbance in the short wavelength flank of the absorption maximum at the wavelength λₘₐₓ₂ is half of the absorbance value at λₘₐₓ₂ and the wavelength λ_{1/10} at which the absorbance in the short wavelength flank of the absorption maximum at the wavelength λₘₐₓ₂ is one tenth of the absorbance value at λₘₐₓ₂ are preferably not more than 50 nm apart. Such a cationic aminoheterocyclic dye preferably has no shorter-wavelength maximum λₘₐₓ₁ down to a wavelength of 350 nm, particularly preferably down to 320 nm, very particularly preferably down to 290 nm.

Preference is given to cationic aminoheterocyclic dyes having an absorption maximum λₘₐₓ₂ of from 410 to 530 nm.

Particular preference is given to cationic aminoheterocyclic dyes having an absorption maximum λₘₐₓ₂ of from 420 to 510 nm.

Very particular preference is given to cationic aminoheterocyclic dyes having an absorption maximum λₘₐₓ₂ of from 430 to 500 nm.

In these cationic aminoheterocyclic dyes, λ_{½} and λ_{1/10}, as defined above, are preferably not more than 40 nm apart, particularly preferably not more than 30 nm apart, very particularly preferably not more than 20 nm apart.

In the case of a write-once optical data carrier according to the invention which is written on and read by means of the light of a red laser, preference is given to cationic aminoheterocyclic dyes whose absorption maximum λₘₐₓ₂ is in the range from 500 to 650 nm, where the wavelength λ_{½} at which the absorbance in the long wavelength flank of the absorption maximum at the wavelength λₘₐₓ₂ is half of the absorbance value at λₘₐₓ₂ and the wavelength λ1/10 at which the absorbance in the long wavelength flank of the absorption maximum at the wavelength λₘₐₓ₂ is one tenth of the absorbance value at λₘₐₓ₂ are preferably not more than 50 nm apart. Such a cationic aminoheterocyclic dye preferably has no longer-wavelength maximum λₘₐₓ₃ up to a wavelength of 750 nm, particularly preferably up to 800 nm, very particularly preferably up to 850 nm.

Preference is given to cationic aminoheterocyclic dyes having an absorption maximum λₘₐₓ₂ of from 530 to 630 nm.

Particular preference is given to cationic aminoheterocyclic dyes having an absorption maximum λₘₐₓ₂ of from 550 to 620 nm.

Very particular preference is given to cationic aminoheterocyclic dyes having an absorption maximum λₘₐₓ₂ of from 580 to 610 nm.

In these cationic aminoheterocyclic dyes, λ_{½} and λ_{1/10}, as defined above, are preferably not more than 40 nm apart, particularly preferably not more than 30 nm apart, very particularly preferably not more than 20 nm apart.

In the case of a write-once optical data carrier according to the invention which is written on and read by means of the light of a infrared laser, preference is given to cationic aminoheterocyclic dyes whose absorption maximum λₘₐₓ₃ is in the range from 650 to 810 nm, where the wavelength λ_{½} at which the absorbance in the long wavelength flank of the absorption maximum at the wavelength λₘₐₓ₃ is half of the absorbance value at λₘₐₓ₃ and the wavelength λ_{1/10} at which the absorbance in the long wavelength flank of the absorption maximum at the wavelength λₘₐₓ₃ is one tenth of the absorbance value at λₘₐₓ₃ are preferably not more than 50 nm apart.

Preference is given to cationic aminoheterocyclic dyes having an absorption maximum λₘₐₓ₃ of from 660 to 790 nm.

Particular preference is given to cationic aminoheterocyclic dyes having an absorption maximum λₘₐₓ₃ of from 670 to 760 nm.

Very particular preference is given to cationic aminoheterocyclic dyes having an absorption maximum λₘₐₓ₃ of from 680 to 740 nm.

In these cationic aminoheterocyclic dyes, λ_{½} and λ_{1/10}, as defined above, are preferably not more than 40 nm apart, particularly preferably not more than 30 nm apart, very particularly preferably not more than 20 nm apart.

The cationic aminoheterocyclic dyes have a molar extinction coefficient ε of >40 000 l/mol cm, preferably >60 000 l/mol cm, particularly preferably >80 000 l/mol cm, very particularly preferably >100 000 l/mol cm, at the absorption maximum λₘₐₓ₂.

The absorption spectra are measured, for example, in solution.

Suitable cationic aminoheterocyclic dyes having the required spectral properties are, in particular, those in which the dipole moment change Δμ=|μ_{g}−μ_{ag}|, i.e. the positive difference between the dipole moments in the ground state and in the first excited state, is very small, preferably <5 D, particularly preferably <2 D. A method of determining such a dipole moment change Ap is described, for example, in F. Wuirthner et al., Angew. Chem. 1997, 109, 2933, and in the literature cited therein. A low solvent-induced wavelength shift (methanol/methylene chloride) is likewise a suitable selection criterion. Preference is given to cationic aminoheterocyclic dyes whose solvent-induced wavelength shift Δλ=|λ_{methylene chloride}−λₘₑₜₕₐₙₒₗ|, i.e. the positive difference between the absorption wavelengths in the solvents methylene chloride and methanol, is <25 nm, particularly preferably <15 nm, very particularly preferably <5 nm.

Some cationic aminoheterocyolic dyes of the formula (I) are known, for example from Synthesis, 1973, 621; Angew. Chem. 2001, 113, 597; thesis by R. Flaig, University of Halle, 1996, Angew. Chem. 2001, 113, 3097.

The invention further provides cationic aminoheterocyclic dyes of the formula (VI),

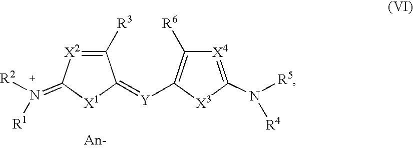

where

X¹ and X³ represent, independently of one another, O or S,

X² represents CR¹⁰ or N,

X⁴ represents CR¹¹ or N,

R³ represents hydrogen, methyl, ethyl, propyl, butyl, chlorine, phenyl, tolyl, methoxyphenyl, xylyl or NR⁸R⁹,

R⁶ represents hydrogen, methyl, ethyl, propyl, butyl, chlorine, phenyl, tolyl, methoxyphenyl, xylyl or NR¹²R¹³ or

R³ and R⁶ together form an —O—, —CH₂— or —C(CH₃)₂— bridge when n=0,

R¹, R², R⁴, R⁵, R⁸, R⁹, R¹² and R¹³ represent, independently of one another, hydrogen, propyl, butyl, cyanoethyl, hydroxyethyl, hydroxypropyl, cyclo-pentyl, cyclohexyl, phenyl, tolyl, methoxyphenyl, xylyl, benzyl, phenethyl or phenylpropyl or

NR¹R², NR⁴R⁵, NR⁸R⁹ and NR¹²R¹³ represent, independently of one another, pyrrolidino, piperazino, N-methyl-piperazino or piperidino,

R¹⁰ and R¹¹ represent, independently of one another, hydrogen, cyano, methyl, ethyl, propyl, butyl, phenyl, tolyl, methoxyphenyl or xylyl,

Y represents ═Y¹—(Y²═Y³)ₙ— or N,

Y¹ to Y³ represent CH,

n represents 0 or 1 and

An⁻ represents an anion.

Preference is given to the members of the pairs X¹; X³, X²; X⁴, R¹; R⁴, R²; R⁵ and R³; R⁶ being identical in each case.

The invention further provides cationic aminoheterocyclic dyes of the formula (VII),

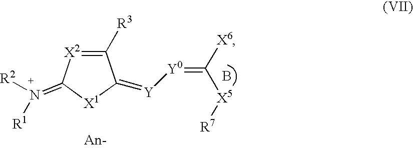

where

X¹ represents O or S,

X² represents CR¹⁰ or N,

R³ represents hydrogen, methyl, ethyl, propyl, butyl, chlorine, phenyl, tolyl, methoxyphenyl, xylyl or NR⁸R⁹,

R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or

NR¹R² and NR⁸R⁹ represent, independently of one another, pyrrolidino, morpholino, pip erazino, N-methyl-pip erazino or pip eridino,

R¹⁰ represents hydrogen, cyano, methyl, ethyl, propyl, butyl, phenyl, tolyl, methoxyphenyl or xylyl,

the ring B together with X⁵ and X⁶ represents, when X⁵ represents N, benzothiazol-2-ylidene, 1,3-thiazol-2-ylidene, benzoxazol-2-ylidene, benz-imidazol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-ylidene or pyrrolin-2-ylidene or, when X⁵—R⁷ represents S, represents 1,3-dithiol-2-ylidene, where the rings mentioned may each be substituted by up to three radicals selected from the group consisting of methyl, ethyl, trifluoromethyl, methoxy, ethoxy, fluorine, chlorine, bromine, cyano, nitro, methoxycarbonyl, ethoxycarbonyl, methylthio, formylamino, acetylamino, propionylamino, phenyl, tolyl, methoxyphenyl, phenoxy, benzoylamino, dimethylamino, diethylamino, dipropylamino, N-methyl-N-phenylamino, pyrrolidino, morpholino or piperidino,

R⁷ represents methyl, ethyl, propyl, butyl, cyanoethyl, hydroxyethyl, hydroxypropyl, allyl, cyclopentyl, cyclohexyl, benzyl, phenethyl or phenylpropyl,

Y represents ═Y¹—(Y²═Y³)ₙ— or N,

Y¹ to Y³ represent CH,

n represents 0 or 1,

Y⁰ represents CH or N and

An⁻ represents an anion.

The invention further provides cationic aminoheterocyclic dyes of the formula (VIII),

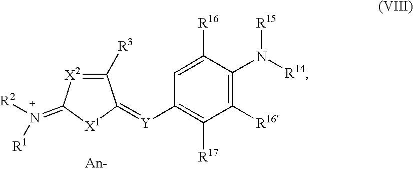

where

X¹ represents O or S,

X² represents CR¹⁰ or N,

R³ represents hydrogen, methyl, ethyl, propyl, butyl, chlorine, phenyl, tolyl, methoxyphenyl, xylyl or NR⁸R⁹,

R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or

NR¹R² and NR⁸R⁹ represent, independently of one another, pyrrolidino, piperazino, N-methyl-piperazino or piperidino,

R¹⁰ represents hydrogen, cyano, methyl, ethyl, propyl, butyl, phenyl, tolyl, methoxyphenyl or xylyl,

R¹⁴ and R¹⁵ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl, phenylpropyl, pyridyl or a radical of the formula

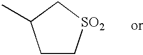

NR¹⁴R¹⁵ represents pyrrolidino, morpholino, piperazino, N-methyl-piperazino or piperidino,

R¹⁶and R^{16′} represent, independently of one another, hydrogen, methyl, ethyl, methoxy, ethoxy or chlorine or

R¹⁶; R¹⁵ and/or R^{16′}; R¹⁴ form a —(CH₂)₂— or —(CH₂)₃— bridge which may be substituted by up to 3 methyl groups,

R¹⁷ represents hydrogen, methyl, ethyl, methoxy, ethoxy, chlorine, cyano, methoxycarbonyl, ethoxycarbonyl, O—CO—R²², NR²³—CO—R²², O—SO₂—R²² or NR²³—SO₂—R²²,

R²² represents methyl, ethyl, trifluoromethyl, cyclopentyl, cyclohexyl, benzyl, methoxy, ethoxy, dimethylamino, diethylamino, methylamino, phenyl, phenoxy, anilino or pyridyl,

R²³ represents hydrogen or methyl,

Y represents ═Y¹—(Y²═Y³)ₙ— or N,

Y¹ to Y³ represent CH,

n represents 0 or 1 and

An⁻ represents an anion.

The light-absorbent compounds described guarantee a sufficiently high reflectivity (>10%) of the optical data carrier in the unwritten state and a sufficiently high absorption for thermal degradation of the information layer on point-wise illumination with focused light if the wavelength of the light is in the range from 360 to 460 nm, from 600 to 680 nm or from 750 to 820 nm. The contrast between written and unwritten points on the data carrier is achieved by the reflectivity change of the amplitude and also the phase of the incident light due to the changed optical properties of the information layer after the thermal degradation.

The cationic aminoheterocyclic dyes are preferably applied to the optical data carrier by spin coating. The cationic aminoheterocyclic dyes can be mixed with one another or with other dyes having similar spectral properties. In particular, cationic dyes containing different anions can also be mixed. The information layer can comprise not only the cationic aminoheterocyclic dyes but also additives such as binders, wetting agents, stabilizers, diluents and sensitizers and also further constituents.

Apart from the information layer, further layers such as metal layers, dielectric layers and protective layers may be present in the optical data carrier. Metals and dielectric layers serve, inter alia, to adjust the reflectivity and the heat absorption/retention. Metals can be, depending on the laser wavelength, gold, silver, aluminium, etc. Examples of dielectric layers are silicon dioxide and silicon nitride. Protective layers are, for example, photocurable surface coatings, adhesive layers and protective films.

The adhesive layers may be pressure-sensitive.

Pressure-sensitive adhesive layers consist mainly of acrylic adhesives. Nitto Denko DA-8320 or DA-8310, disclosed in the patent JP-A 11-273147, can, for example, be used for this purpose.

The invention further provides a write-once optical data carrier comprising a preferably transparent substrate to whose surface at least one light-writable information layer, if desired a reflection layer and/or, if desired, a protective layer have been applied, which can be written on and read by means of blue, red or infrared light, preferably laser light, where the information layer comprises at least one of the abovementioned light-absorbent compounds and, if desired, a binder, wetting agents, stabilizers, diluents and sensitizers and also further constituents. Alternatively, the structure of the optical data carrier can

comprise a preferably transparent substrate to whose surface at least one light-writable information layer, if desired a reflection layer and, if desired, an adhesive layer and a further preferably transparent substrate have been applied, or

comprise a preferably transparent substrate to whose surface if desired a reflection layer, at least one light-writable information layer, if desired an adhesive layer and a transparent covering layer have been applied.

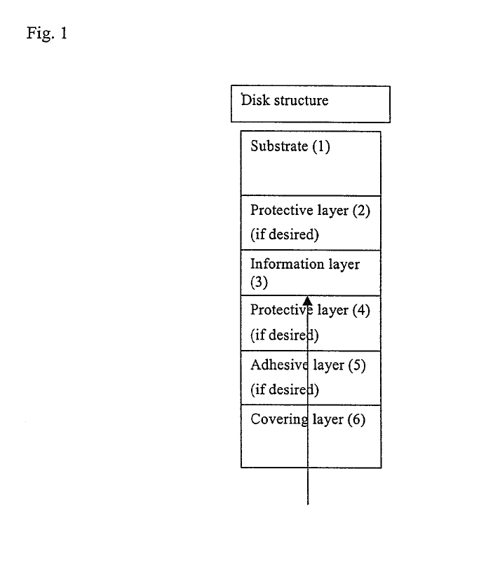

The optical data carrier has, for example, the following layer structure (cf. FIG. 1): a transparent substrate (1), if desired a protective layer (2) an information layer (3), if desired a protective layer (4), if desired an adhesive layer (5), a covering layer (6).

The structure of the optical data carrier preferably:

comprises a preferably transparent substrate (1) to whose surface at least one light-writeable information layer (3) which can be written on by means of light, preferably laser light, if desired a protective layer (4), if desired an adhesive layer (5) and a transparent covering layer (6) have been applied.

comprises a preferably transparent substrate (1) to whose surface a protective layer (2), at least one information layer (3) which can be written on by means of light, preferably laser light, if desired an adhesive layer (5) and a transparent covering layer (6) have been applied.

comprises a preferably transparent substrate (1) to whose surface a protective layer (2) if desired, at least one information layer (3) which can be written on by means of light, preferably laser light, if desired a protective layer (4), if desired an adhesive layer (5) and a transparent covering layer (6) have been applied.

comprises a preferably transparent substrate (1) to whose surface at least one information layer (3) which can be written on by means of light, preferably laser light, if desired an adhesive layer (5) and a transparent covering layer (6) have been applied.

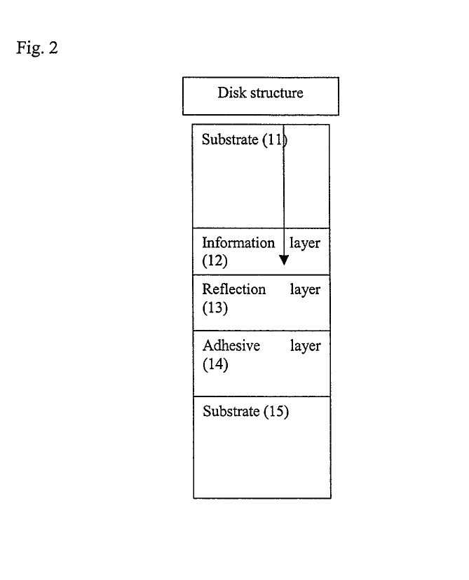

Alternatively, the optical data carrier has, for example, the following layer structure (cf. FIG. 2): a preferably transparent substrate (11), an information layer (12), if desired a reflection layer (13), if desired an adhesive layer (14), a further preferably transparent substrate (15).

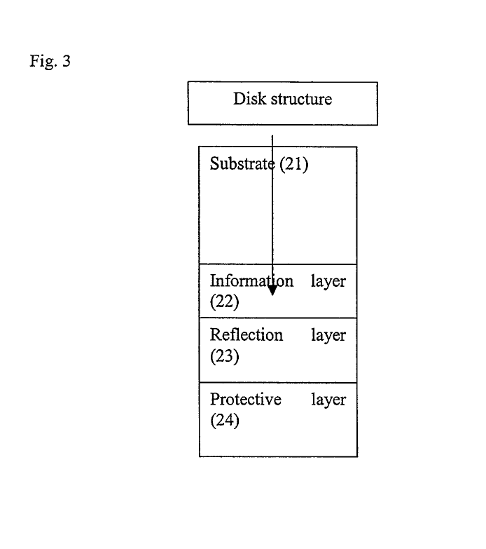

Alternatively, the optical data carrier has, for example, the following layer structure (cf. FIG. 3): a preferably transparent substrate (21), an information layer (22), if desired a reflection layer (23), a protective layer (24).

The invention further provides optical data carriers according to the invention which have been written on by means of blue, red or infrared light, in particular laser light.

The following examples illustrate the subject-matter of the invention.

### EXAMPLES

### Example 1

A mixture of 2.5 g of 2-morpholino-4-phenyl-thiazole, 0.5 g of trimethyl ortho-formate, 0.5 g of lithium perchlorate and 10 ml of acetic anhydride was stirred at 90° C. for 3 hours. After cooling, the mixture was filtered with suction, the solid was washed with 10 ml of ethyl acetate and dried. This gave 1.6 g (53% of theory) of blue powder of the formula

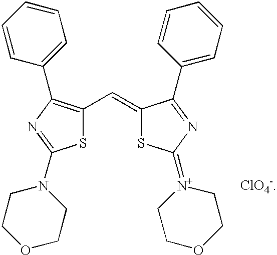

m.p.=265-270° C.

λₘₐₓ (methanol)=581 nm

λₘₐₓ (methylene chloride)=587 nm

ε=102 800 l/mol cm

λ_{½}-λ_{1/10} (short wavelength flank)=42 nm

λ_{½}-λ_{1/10} (long wavelength flank)=14.5 nm

Solubility: >2% in TFP

### Example 2

1.4 g of 5-dimethylaminofuran-2-carbaldehyde and 1.74 g of 1,3,3-trimethyl-2-methylene-3H-indole were stirred in a mixture of 5 ml of glacial acetic acid and 1 ml of acetic anhydride at 40° C. for 2 hours. After cooling, the mixture was poured into 80 ml of water in which 2.6 g of sodium tetrafluoroborate had been dissolved. The mixture was filtered with suction, the solid was washed with water and dried. This gave 1.6 g (42% of theory) of a red powder of the formula

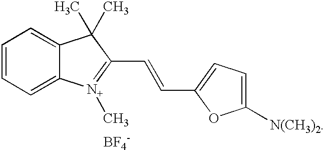

m.p.=218° C.

λₘₐₓ (methanol)=551 nm

ε=87 670 l/mol cm

λ_{½}-λ_{1/10} (short wavelength flank)=41 nm

λ_{½}-λ_{1/10} (long wavelength flank)=30 nm

Solubility: >2% in TFP

Glass-like film

### Example 3

2.4 g of 2-dibutylaminothiazole-5-carbaldehyde and 1.74 g of 1,3,3-trimethyl-2-methylene-3H-indole were stirred in 10 ml of acetic anhydride at 90° C. for 2 hours. After cooling, the mixture was poured into 100 ml of water. 2.0 g of lithium perchlorate were added. The dye which had precipitated was filtered off with suction, washed with water and dried. This gave 4.1 g (82% of theory) of a red powder of the formula

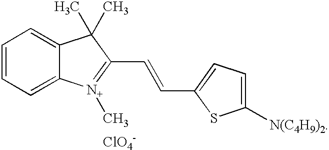

m.p.=185-190° C.

λₘₐₓ (methanol)=528 nm

ε=68 555 l/mol cm

λₘₐₓ (methylene chloride)=543 nm

ε=98 554 l/mol cm

λ_{½}-λ_{1/10} (short wavelength flank)=34 nm

λ_{½}-λ_{1/10} (long wavelength flank)=14 nm

Solubility: >2% in TFP

Glass-like film

### Example 4

A mixture of 2.5 g of 2-morpholino-4-phenylthiazole, 1.5 g of 4-dimethylamino-benzaldehyde, 1.1 g of lithium perchlorate and 20 ml of acetic anhydride was stirred at 80-90° C. for 2 hours. After cooling, the mixture was filtered with suction and the solid was washed with 20 ml of ethyl acetate and dried. This gave 3.7 g (77% of theory) of blue powder of the formula

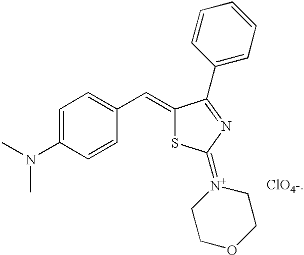

m.p.=218-220° C.

λₘₐₓ (methylene chloride)=601 nm

ε=104 970 l/mol cm

λ_{½}-λ_{1/10} (short wavelength flank)=42 nm

λ_{½}-λ_{1/10} (long wavelength flank)=19 nm

Solubility: >2% in TFP

Further suitable cationic aminoheterocyclic dyes are shown in the following tables:

Dyes of the formula (VI)

| Ex- ample | 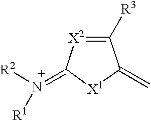 | Y | 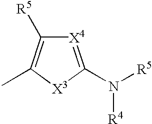 | An⁻ | λₘₐₓ/ nm¹⁾ | ε/ l/mol cm | λ_{1/2}- λ_{1/10}/ nm | Δλ²⁾/ nm |
|---|---|---|---|---|---|---|---|---|
| 5 | 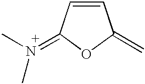 | CH | 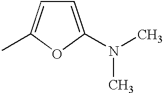 | I⁻ | 583³⁾ | 125890 | | |
| 6 | 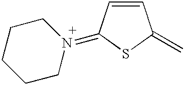 | CH | 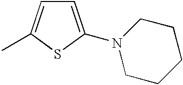 | ClO₄⁻ | 592³⁾ | 131825 | | |
| 7 | 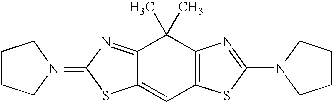 | | | BF₄⁻ | 616 | 37153 | 50⁴⁾27⁵⁾ | |
| 8 | 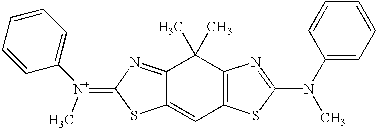 | | | BF₄⁻ | 623 | 43650 | | |
| 9 | 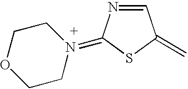 | CH | 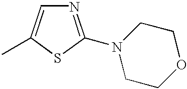 | ClO₄⁻ | 552 | 85114 | 14⁵⁾ | 9 |
| 10 | 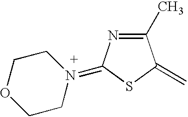 | CH | 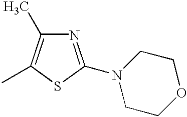 | ClO₄⁻ | 554 | | | |
| 11 | 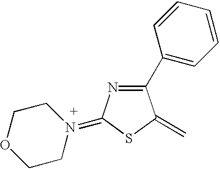 | CH | 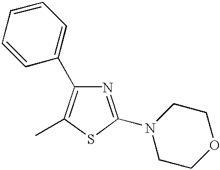 | ClO₄⁻ | 586 | 89125 | | |
| 12 | 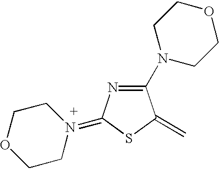 | CH | 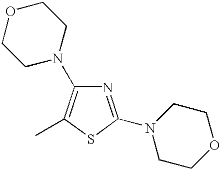 | ClO₄⁻ | 534 | 56234 | | |
| 13 | 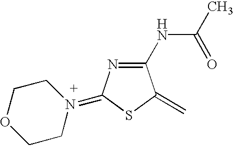 | CH | 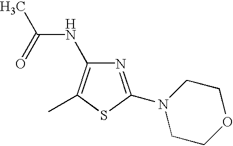 | ClO₄⁻ | 554 | | | |
| 14 | 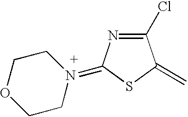 | CH | 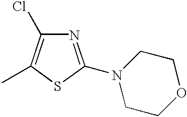 | ClO₄⁻ | 572 | | | |
| 15 | 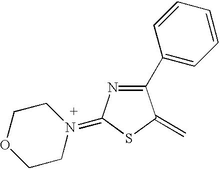 | CH | 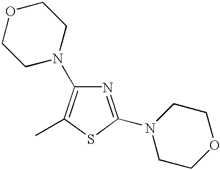 | ClO₄⁻ | 516 | 45709 | 25⁵⁾ | 19 |
| 16 | 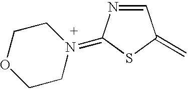 | CH—CH═CH | 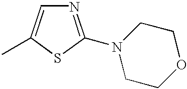 | ClO₄⁻ | 642 | 120226 | | |
| 17 | 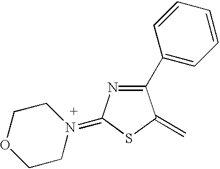 | CH—CH═CH | 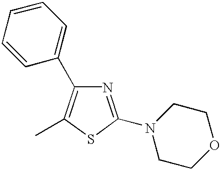 | ClO₄⁻ | 680 | 169825 | | |
| 18 | 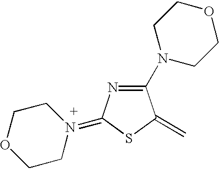 | CH—CH═CH | 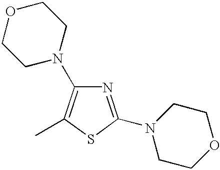 | ClO₄⁻ | 618 | 125890 | | |
| 19 | 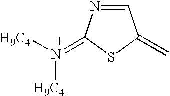 | CH | 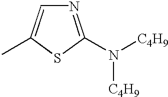 | CH₃SO₃⁻ | 555 | | | |
| 20 | 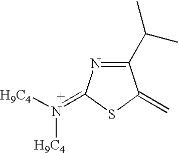 | CH | 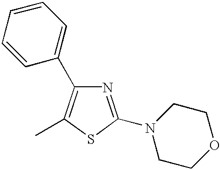 | ClO₄⁻ | 554 | | | |
| 21 | 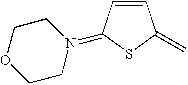 | 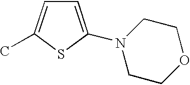 | 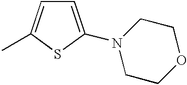 | ClO₄⁻ | 615 | 93325 | | |
| 22 | 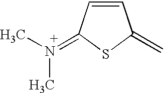 | 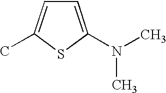 | 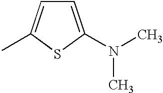 | ClO₄⁻ | 697 | 77625 | | |
| 23 | 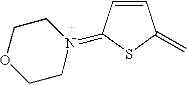 | 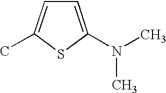 | 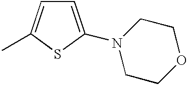 | ClO₄⁻ | 612 | 79430 | | |
| 24 | 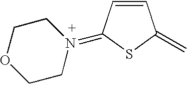 | 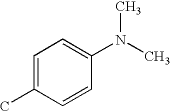 | 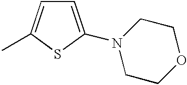 | ClO₄⁻ | 605 | 107150 | | |

¹⁾in methylene chloride unless indicated otherwise.
²⁾Δλ = |λ_{methylene chloride} - λₘₑₜₕₐₙₒₗ|
³⁾in nitromethane
⁴⁾on the short wavelength flank
⁵⁾on the long wavelength flank

Dyes of the formula (VII)

| Ex- ample | 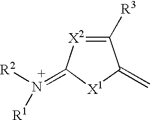 | Y | 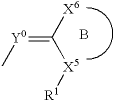 | An⁻ | λₘₐₓ/ nm¹⁾ | ε/ l/mol cm | λ_{1/2}- λ_{1/10}/ nm | Δλ²⁾/ nm |
|---|---|---|---|---|---|---|---|---|
| 25 | 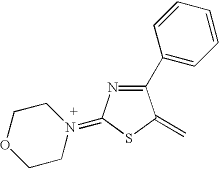 | CH | 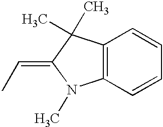 | ClO₄⁻ | | | | |
| 26 | 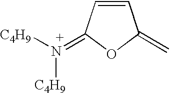 | CH | 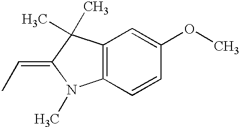 | ClO₄⁻ | | | | |
| 27 | 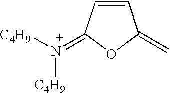 | CH | 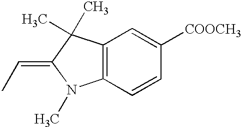 | ClO₄⁻ | | | | |
| 28 | 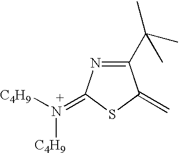 | CH | 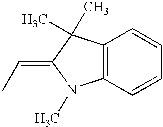 | ClO₄⁻ | | | | |
| 29 | 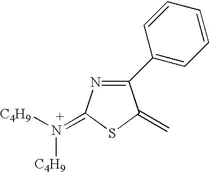 | CH | 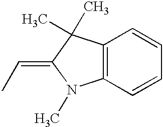 ClO₄⁻ | | | | | |
| 30 | 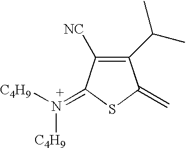 | CH | 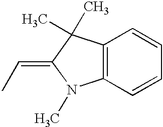 | ClO₄⁻ | | | | |
| 31 | 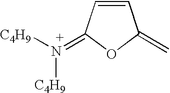 | CH | 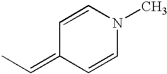 | ClO₄⁻ | | | | |
| 32 | 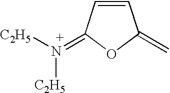 | CH | 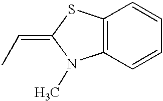 | 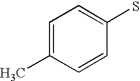 | | | | |
| 33 | 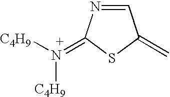 | CH | 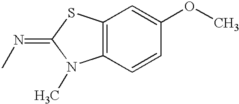 | ClO₄⁻ | | | | |
| 34 | 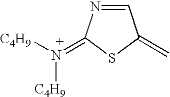 | CH | 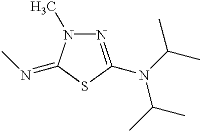 | ClO₄⁻ | | | | |
| 35 | 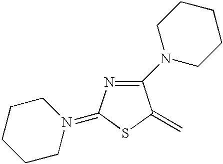 | CH | 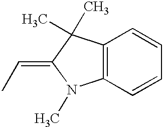 | ClO₄⁻ | | | | |
| 36 | 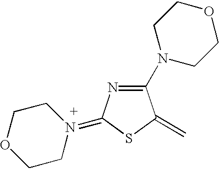 | N | 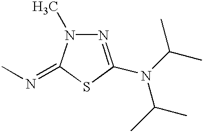 | ClO₄⁻ | | | | |
| 37 | 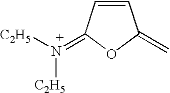 | N | 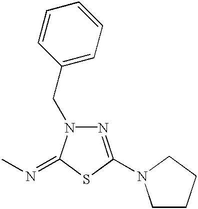 | BF₄⁻ | | | | |
| 38 | 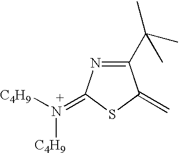 | CH | 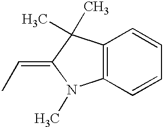 | ClO₄⁻ | 560 | | | |
| 39 | 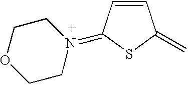 | 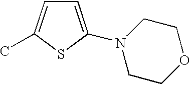 | 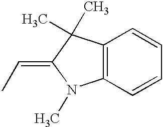 | ClO₄⁻ | 625 | 131825 | | |

¹⁾in methylene chloride unless indicated otherwise.
²⁾Δλ = |λ_{methylene chloride} - λₘₑₜₕₐₙₒₗ|
³⁾in nitromethane
⁴⁾on the short wavelength flank
⁵⁾on the long wavelength flank

Dyes of the formula (VIII)

| Ex- ample | 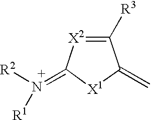 | Y | 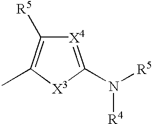 | An⁻ | λₘₐₓ/ nm¹⁾ | ε/ l/mol cm | λ_{1/2}- λ_{1/10}/ nm | Δλ²⁾/ nm |
|---|---|---|---|---|---|---|---|---|
| 40 | 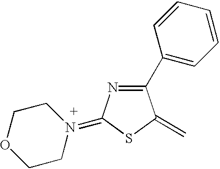 | CH | 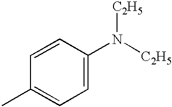 | ClO₄⁻ | 606 | | | |
| 41 | 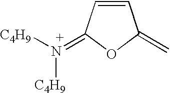 | CH | 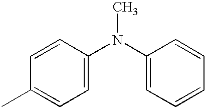 | ClO₄⁻ | | | | |
| 42 | 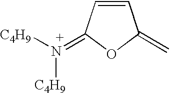 | CH | 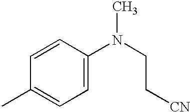 | ClO₄⁻ | | | | |
| 43 | 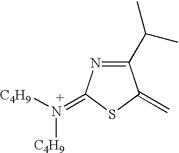 | CH | 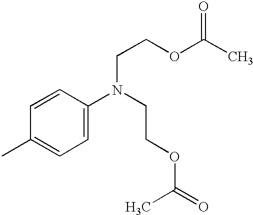 | ClO₄⁻ | | | | |
| 44 | 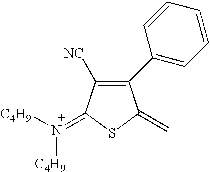 | CH | 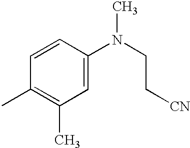 | ClO₄⁻ | | | | |
| 45 | 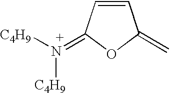 | CH | 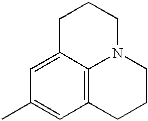 | ClO₄⁻ | | | | |
| 46 | 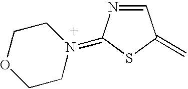 | CH | 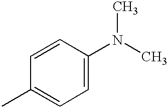 | ClO₄⁻ | 584 | 123027 | 14⁵⁾ | 10 |
| 47 | 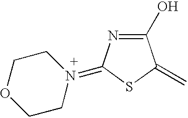 | CH | 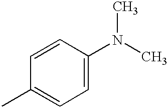 | ClO₄⁻ | 413 | 34674 | | |
| 48 | 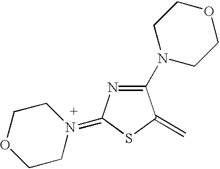 | CH | 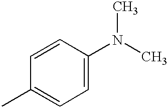 | ClO₄⁻ | 512 | 51286 | 33⁴⁾29⁵⁾ | 22 |
| 49 | 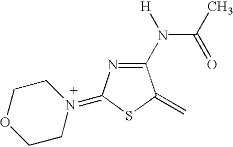 | CH | 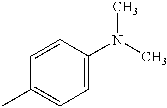 | ClO₄⁻ | 550 | | | |
| 50 | 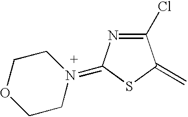 | CH | 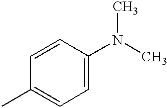 | ClO₄⁻ | 600 | | | |
| 51 | 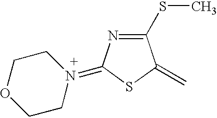 | CH | 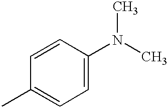 | ClO₄⁻ | 591 | 104713 | | |
| 52 | 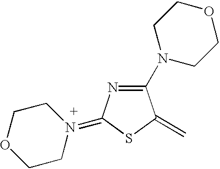 | N | 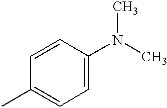 | PF₆⁻ | 546 | 30903 | | |
| 53 | 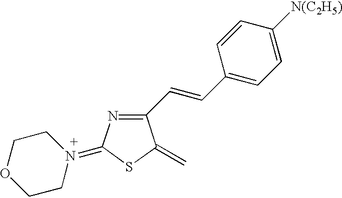 | CH | 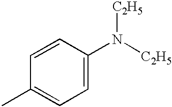 | ClO₄⁻ | 710 | 104713 | | |
| 54 | 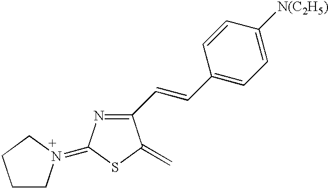 | CH | 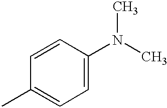 | ClO₄⁻ | 692 | 97724 | | |
| 55 | 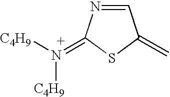 | CH | 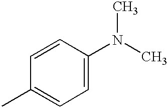 | ClO₄⁻ | 555 | | | |
| 56 | 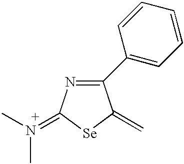 | CH | 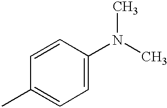 | ClO₄⁻ | 596 | 104715 | | |
| 57 | 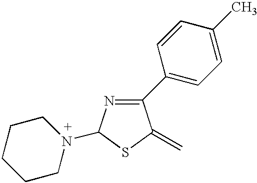 | CH—CH═CH | 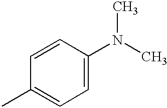 | ClO₄⁻ | 694 | 165880 | | |

¹⁾in methylene chloride unless indicated otherwise.
²⁾Δλ = |λ_{methylene chloride} - λₘₑₜₕₐₙₒₗ|
³⁾in nitromethane
⁴⁾on the short wavelength flank
⁵⁾on the long wavelength flank

### Example 58

A 2% strength by weight solution of the dye from Example 1, in 2,2,3,3-tetrafluoropropanol was prepared at room temperature. This solution was applied by means of spin coating to a pregrooved polycarbonate substrate. The pregrooved polycarbonate substrate had been produced as a disk by means of injection moulding. The dimensions of the disk and the groove structure corresponded to those customarily used for DVD-Rs. The disk with the dye layer as information carrier was coated with 100 nm of silver by vapour deposition. A UV-curable acrylic coating composition was subsequently applied by spin coating and cured by means of a UV lamp. The disk was tested by means of a dynamic writing test apparatus constructed on an optical tester bench comprising a diode laser (λ=656 nm) for generating linearly polarized light, a polarization-sensitive beam splitter, a λ/4 plate and a movably suspended collecting lens having a numerical aperture NA=0.6 (actuator lens). The light reflected from the reflection layer of the disk was taken out from the beam path by means of the abovementioned polarization-sensitive beam splitter and focused by means of an astigmatic lens onto a four-quadrant detector. At a linear velocity V=3.5 m/s and a writing power P_{w}=8 mW, a signal/noise ratio C/N=36 dB was measured. The writing power was applied as an oscillating pulse sequence, with the disk being irradiated alternately for 1 μs with the abovementioned writing power P_{w} and for 4 μs with the reading power Pᵣ≈0.6 mW. The disk was irradiated with this oscillating pulse sequence until it had rotated once. The marking produced in this way was then read using the reading power Pᵣ and the abovementioned signal/noise ratio C/N was measured.

### Example 59

Using an analogous procedure, a dye layer consisting of 66.7% by weight of the dye of the formula

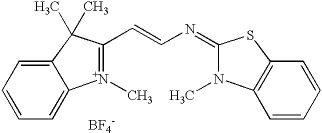

and 33.3% by weight of the dye from Example 1 was applied to an equivalent substrate, 120 nm of gold were applied by vapour deposition and 200 nm of SiO were subsequently applied on top of the gold layer by vapour deposition. A layer of a UV-curable acrylic surface coating composition was subsequently applied thereto. Under analogous measurement conditions, a C/N=42 dB was obtained at a writing power P_{w}=21 mW.

1. Optical data carrier comprising a preferably transparent substrate which may, if desired, have previously been coated with one or more reflection layers and to whose surface a light-writeable information layer, if desired one or more reflection layers and if desired a protective layer or a farther substrate or a covering layer have been applied, which can be written on or read by means of blue, red or infrared light, preferably laser light, where the information layer comprises a light-absorbent compound and, if desired, a binder, characterized in that at least one cationic aminoheterocyclic dye is used as light-absorbent compound.

2. Optical data carrier according to claim 1, characterized in that the cationic aminoheterocyclic dye has the formula I 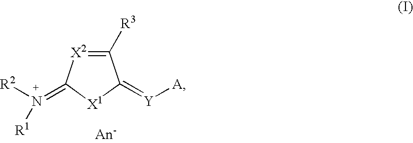
where
X¹ represents O or S,
X² represents CR¹⁰ or N,
R³ represents hydrogen, C₁-C₆-alkyl, halogen, hydroxy, C₆-C₁₀-aryl, NR⁸R⁹ or —CH═Y—A,
R¹, R², R⁸ and R⁹ represent, independently of one another, hydrogen, C₁-C₆-alkyl, C₅-C₇-cycloalkyl, C₆-C₁₀-aryl or C₇-C₁₅-aralkyl or
NR¹R² and NR⁸R⁹ represent, independently of one another, pyrrolidino, morpholino, piperazino or piperidino,
R¹⁰ represents hydrogen, cyano, C₁-C₆-alkyl, halogen or C₆-C₁₀-aryl,
Y represents ═Y¹—(Y²═Y³)ₙ—,
Y¹ to Y³ represent, independently of one another, N or C—R¹⁸, where, if n=1, the radicals R¹⁸ of Y¹; Y², Y¹; Y³ or Y²; Y³ may in each case form a bridge,
n represents 0 or 1,
R¹⁸ represents hydrogen, cyano or C₁-C₃-alkyl,
A represents a radical of the formula 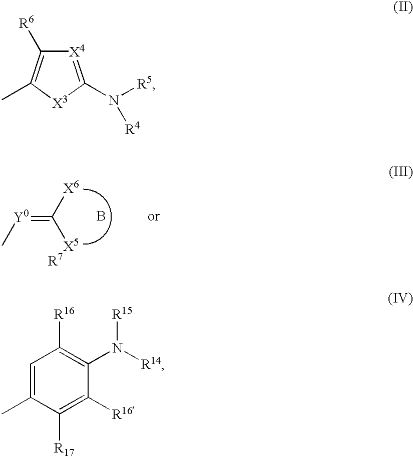
where
X³ represents O or S,
X⁴ represents CR¹¹ or N,
R⁶ represents hydrogen, C₁-C₆-alkyl, halogen, hydroxy, C₆-C₁₀-aryl or NR¹²R¹³ or
R³ and R⁶ may form an —O—, —CH₂— or —C(CH₃)₂— bridge or a bridge of the formula 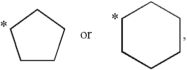
, where two single bonds go out from the asterisked (*) carbon atom,
R⁴, R⁵, R¹² and R¹³ represent, independently of one another, hydrogen, C₁-C₆-alkyl, C₅-C₇-cycloalkyl, C₆-C₁₀-aryl or C₇-C₁₅-aralkyl or
NR⁴R⁵ and NR¹²R¹³ represent, independently of one another, pyrrolidino, morpholino, piperazino or piperidino,
R¹¹ represents hydrogen, cyano, C₁-C₆-alkyl, halogen or C₆-C₁₀-aryl,
X⁵ represents nitrogen or
X⁵—R⁷ represents S,
X⁶ represents O, S, N—R¹⁹, CR²⁰ or CR²⁰R²¹,
Y⁰ represents N or C—R¹⁸,
R⁷ and R¹⁹ represent, independently of one another, C₁-C₁₆-alkyl, C₃-C₆-alkenyl, C₅-C₇-cycloalkyl or C₇-C₁₆-aralkyl,
R²⁰ and R²¹ represent, independently of one another, C₁-C₄-alkyl or
CR²⁰R²¹ represents a bivalent radical of the formula 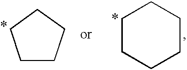
where the two bonds go out from the asterisked (*) ring atom,
B together with X⁵, X⁶ and the carbon atom between them represents a five- or six-membered aromatic or pseudoaromatic or partially hydro-genated heterocyclic ring which can contain from 1 to 4 heteroatoms and/or may be benzo- or naphtho-fused and/or substituted by nonionic radicals,
R¹⁴ and R¹⁵ represent, independently of one another, hydrogen, C₁-C₁₆-alkyl, C₄-C₇-cycloalkyl, C₇-C₁₆-aralkyl, C₆-C₁₀-aryl or a heterocyclic radical or
NR¹⁴R¹⁵ represents a five- or six-membered saturated ring which is bound via N and may additionally contain an N or O atom and/or be substituted by nonionic radicals,
R¹⁶ and R^{16′} represent, independently of one another, hydrogen, C₁-C₁₆-alkyl, C₁-C₁₆-alkoxy or halogen or
R¹; R¹⁴ and/or R^{16′}; R¹⁵ form a two- or three-membered bridge which may contain an O or N atom and/or be substituted by nonionic radicals,
R¹⁷ represents hydrogen, C₁-C₁₆-alkyl, C₁-C₁₆-alkoxy, halogen, cyano, C₁-C₄-alkoxycarbonyl, O—CO—R²², NR²³—CO—R²², O—SO₂—R²² or NR²³—SO₂—R²²,
R²² represents hydrogen, C₁-C₁₆-alkyl, C₄-C₇-cycloalkyl, C₇-C₁₆-aralkyl, C₁-C₁₆-alkoxy, mono- or bis-C₁-C₁₆-alkylamino, C₆-C₁₀-aryl, C₆-C₁₀-aryloxy, C₆-C₁₀-arylamino or a heterocyclic radical,
R²³ represents hydrogen or C₁-C₄-alkyl,
and
An⁻ represents an anion.

3. Optical data carrier according to claim 1 or 2, characterized in that, in formula (I)
the ring B of the formula 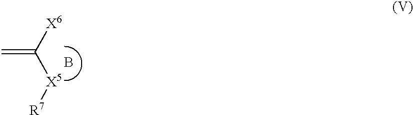
represents benzothiazol-2-ylidene, 1,3-thiazol-2-ylidene, isothiazol-3-ylidene, benzoxazol-2-ylidene, 1,3-oxazol-2-ylidene, isoxazol-3-ylidene, benz-imidazol-2-ylidene, imidazol-2-ylidene, pyrazol-5-ylidene, 1,3,4-thiadiazol-2-ylidene, 1,3,4-oxadiazol-2-ylidene, 1,2,4-thiadiazol-5-ylidene, 1,3,4-triazol-2-ylidene, 1,3-dithiol-2-ylidene, 3-H-indol-2-ylidene, dihydropyridin-2- or -4-ylidene, dihydroquinolin-2- or -4-ylidene, 1,3-thiazolin-2-ylidene, 1,3-oxa-zolin-2-ylidene, imidazolin-2-ylidene or pyrrolin-2-ylidene, where the rings mentioned may each be substituted by C₁-C₆-alkyl, C₁-C₆-alkoxy, fluorine, chlorine, bromine, iodine, cyano, nitro, C₁-C₆-alkoxycarbonyl, C₁-C₆-alkylthio, C₁-C₆-acylamino, C₆-C₁₀-aryl, C₆-C₁₀-aryloxy, C₆-C₁₀-aryl-carbonylamino, mono- or di-C₁-C₆-alkylamino, N—C₁-C₆-alkyl-N—C₆-C₁₀-aryl-amino, pyrrolidino, morpholino or piperidino.

4. Optical data carrier according to claim 1 or 2, characterized in that the cationic aminoheterocyclic dye used is one of the formula (VI) 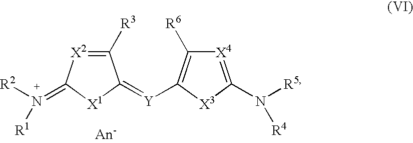
where
X¹ and X³ represent, independently of one another, O or S,
X² represents CR¹⁰ or N,
X⁴ represents CR¹¹ or N,
R³ represents hydrogen, methyl, ethyl, propyl, butyl, chlorine, phenyl, tolyl, methoxyphenyl, xylyl or NR⁸R⁹,
R⁶ represents hydrogen, methyl, ethyl, propyl, butyl, chlorine, phenyl, tolyl, methoxyphenyl, xylyl or NR¹²R¹³ or
R³ and R⁶ together form an —O—, —CH₂— or —C(CH₃)₂— bridge when n=0,
R¹, R², R⁴, R⁵, R⁸, R⁹, R¹² and R¹³ represent, independently of one another, hydrogen, methyl, ethyl, propyl, butyl, cyanoethyl, hydroxyethyl, hydroxypropyl, cyclopentyl, cyclohexyl, phenyl, tolyl, meth-oxyphenyl, xylyl, benzyl, phenethyl or phenylpropyl or
NR¹R², NR⁴R⁵, NR⁸R⁹ and NR¹²R¹³ represent, independently of one another, pyrrolidino, morpholino, piperazino, N-methyl-piperazino or piperidino,
R¹⁰ and R¹¹ represent, independently of one another, hydrogen, cyano, methyl, ethyl, propyl, butyl, phenyl, tolyl, methoxyphenyl or xylyl,
Y represents ═Y¹—(Y²═Y³)ₙ— or N,
Y¹ to Y³ represent CH,
n represents 0 or 1 and
An⁻ represents an anion.

5. Optical data carrier according to one or more of claims 1 to 3, characterized in that the cationic aminoheterocyclic dye used is one of the formula (VII), 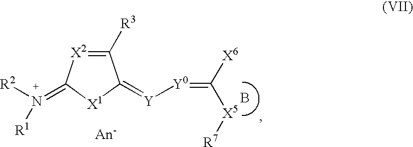
where
X¹ represents O or S,
X² represents CR¹⁰ or N,
R³ represents hydrogen, methyl, ethyl, propyl, butyl, chlorine, phenyl, tolyl, methoxyphenyl, xylyl or NR⁸R⁹,
R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R² and NR⁸R⁹ represent, independently of one another, pyrrolidino, morpholino, piperazino, N-methyl-piperazino or piperidino,
R¹⁰ represents hydrogen, cyano, methyl, ethyl, propyl, butyl, phenyl, tolyl, methoxyphenyl or xylyl,
he ring B together with X⁵ and X⁶ represents, when X⁵ represents N, benzothiazol-2-ylidene, 1,3-thiazol-2-ylidene, benzoxazol-2-ylidene, benzimidazol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-ylidene or pyrrolin-2-ylidene or, when X⁵—R⁷ represents S, represents 1,3-dithiol-2-ylidene, where the rings mentioned may each be substituted by up to three radicals selected from the group consisting of methyl, ethyl, trifluoromethyl, methoxy, ethoxy, fluorine, chlorine, bromine, cyano, nitro, methoxy-carbonyl, ethoxycarbonyl, methylthio, formylamino, acetylamino, propionylamino, phenyl, tolyl, methoxyphenyl, phenoxy, benzoyl-amino, dimethylamino, diethylamino, dipropylamino, N-methyl-N-phenylamino, pyrrolidino, morpholino or piperidino,
R⁷ represents methyl, ethyl, propyl, butyl, cyanoethyl, hydroxyethyl, hydroxypropyl, allyl, cyclopentyl, cyclohexyl, benzyl, phenethyl or phenylpropyl,
Y represents ═Y¹—(Y²═Y³)ₙ— or N,
Y¹ to Y³ represent CH,
n represents 0 or 1,
Y⁰ represents CH or N and
An⁻ represents an anion.

6. Optical data carrier according to claim 1 or 2, characterized in that the cationic aminoheterocyclic dye used has the formula (VIII), 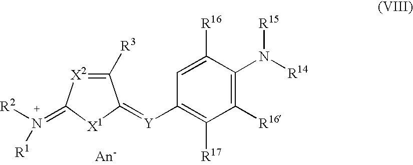
where
X¹ represents O or S,
X² represents CR¹⁰ or N,
R³ represents hydrogen, methyl, ethyl, propyl, butyl, chlorine, phenyl, tolyl, methoxyphenyl, xylyl or NR⁸R⁹,
R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R² and NR⁸R⁹ represent, independently of one another, pyrrolidino, morpholino, piperazino, N-methyl-piperazino or piperidino,
R¹⁰ represents hydrogen, cyano, methyl, ethyl, propyl, butyl, phenyl, tolyl, methoxyphenyl or xylyl,
R¹⁴ and R¹⁵ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl, phenylpropyl, pyridyl or a radical of the formula 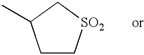
NR¹⁴R¹⁵ represents pyrrolidino, morpholino, piperazino, N-methyl-piperazino or piperidino,
R¹⁶ and R^{16′} represent, independently of one another, hydrogen, methyl, ethyl, methoxy, ethoxy or chlorine or
R¹⁶; R¹⁵ and/or R^{16′}; R¹⁴ form a —(CH₂)₂— or —(CH₂)₃— bridge which may be substituted by up to 3 methyl groups,
R¹⁷ represents hydrogen, methyl, ethyl, methoxy, ethoxy, chlorine, cyano, methoxycarbonyl, ethoxycarbonyl, O—CO—R²², NR²³—CO—R²², O—SO₂—R²² or NR²³—SO₂—R²²,
R²² represents methyl, ethyl, trifluoromethyl, cyclopentyl, cyclohexyl, benzyl, methoxy, ethoxy, dimethylamino, diethylamino, methylamino, phenyl, phenoxy, anilino or pyridyl,
R²³ represents hydrogen or methyl,
Y represents ═Y¹—(Y²═Y³)ₙ— or N,
Y¹ to Y³ represent CH,
n represents 0 or 1 and
An⁻ represents an anion.

7. Optical data carrier according to claim 1, characterized in that the cationic aminoheterocyclic dye has the formula (I) 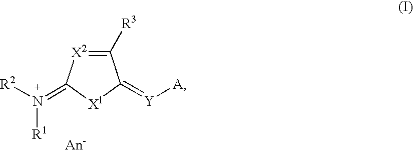
where
X¹ represents O, S or Se,
X² represents CR¹⁰ or N,
R³ represents hydrogen, C₁-C₆-alkyl, halogen, hydroxy, C₆-C₁₀-aryl, NR⁸R⁹ or —CH═Y—A,
R¹, R², R⁸ and R⁹ represent, independently of one another, hydrogen, C₁-C₆-alkyl, C₅-C₇-cycloalkyl, C₆-C₁₀-aryl or C₇-C₁₅-aralkyl or
NR¹R² and NR⁸R⁹ represent, independently of one another, pyrrolidino, morpholino, piperazino or piperidino,
R¹⁰ represents hydrogen, cyano, C₁-C₆-alkyl, halogen or C₆-C₁₀-aryl,
Y represents ═Y¹—(Y²═Y³)ₙ—,
Y¹ to Y³ represent, independently of one another, N or C—R¹⁸, where, if n=1, the radicals R¹⁸ of Y¹; Y², Y¹; Y³ or Y²; Y³ may in each case form a bridge,
n represents 0 or 1,
R¹⁸ represents hydrogen, cyano, C₁-C₃-alkyl or a radical A,
A represents a radical of the formula 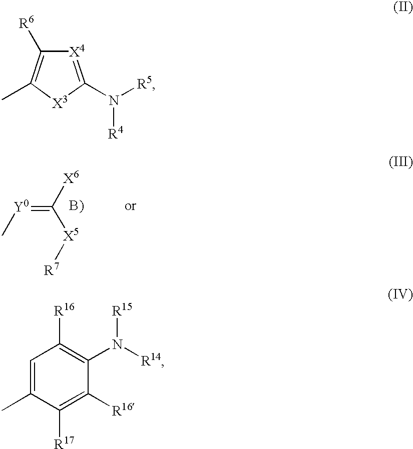
where
X³ represents O, S or Se,
X⁴ represents CR¹¹ or N,
R⁶ represents hydrogen, C₁-C₆-alkyl, halogen, hydroxy, C₆-C₁₀-aryl or NR¹²R¹³ or
R³ and R⁶ may form an —O—, —CH₂— or —C(CH₃)₂— bridge or a bridge of the formula 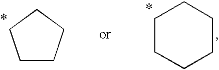
, where two single bonds go out from the asterisked (*) carbon atom,
R⁴, R⁵, R¹² and R¹³ represent, independently of one another, hydrogen, C₁-C₆-alkyl, C₅-C₇-cycloalkyl, C₆-C₁₀-aryl or C₇-C₁₅-aralkyl or
NR⁴R⁵ and NR¹²R¹³ represent, independently of one another, pyrrolidino, morpholino, piperazino or piperidino,
R¹¹ represents hydrogen, cyano, C₁-C₆-alkyl, halogen or C₆-C₁₀-aryl,
X⁵ represents nitrogen or
X⁵—R⁷ represents S,
X⁶ represents O, S, N—R¹⁹, CR²⁰ or CR²⁰R²¹,
Y⁰ represents N or C—R¹⁸,
R⁷ and R¹⁹ represent, independently of one another, C₁-C₁₆-alkyl, C₃-C₆-alkenyl, C₅-C₇-cycloalkyl or C₇-C₁₆-aralkyl,
R²⁰ and R²¹ represent, independently of one another, C₁-C₄-alkyl or
CR²⁰R²¹ represents a bivalent radical of the formula 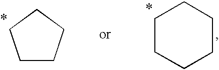
where the two bonds go out from the asterisked (*) ring atom,
B together with X⁵, X⁶ and the carbon atom between them represents a five- or six-membered aromatic or pseudoaromatic or partially hydro-genated heterocyclic ring which can contain from 1 to 4 heteroatoms and/or may be benzo- or naphtho-fused and/or substituted by nonionic radicals,
R¹⁴ and R¹⁵ represent, independently of one another, hydrogen, C₁-C₁₆-alkyl, C₄-C₇-cycloalkyl, C₇-C₁₆-aralkyl, C₆-C₁₀-aryl or a heterocyclic radical or
NR¹⁴R¹⁵ represents a five- or six-membered saturated ring which is bound via N and may additionally contain an N or O atom and/or be substituted by nonionic radicals,
R¹⁶ and R^{16′} represent, independently of one another, hydrogen, C₁-C₁₆-alkyl, C₁-C₁₆-alkoxy or halogen or
R¹⁶; R¹⁴ and/or R^{16′}; R¹⁵ form a two- or three-membered bridge which may contain an O or N atom and/or be substituted by nonionic radicals,
R¹⁷ represents hydrogen, C₁-C₁₆-alkyl, C₁-C₁₆-alkoxy, halogen, cyano, C₁-C₄-alkoxycarbonyl, O—CO—R²², NR²³—CO—R²², O—SO₂—R²² or NR²³—SO₂—R²²,
R²² represents hydrogen, C₁-C₁₆-alkyl, C₄-C₇-cycloalkyl, C₇-C₁₆-aralkyl, C₁-C₁₆-alkoxy, mono- or bis-C₁-C₁₆-alkylamino, C₆-C₁₀-aryl, C₆-C₁₀-aryloxy, C₆-C₁₀-arylamino or a heterocyclic radical,
R²³ represents hydrogen or C₁-C₄-alkyl,
and
An⁻ represents an anion.

8. Optical data carrier according to claim 1 or 7, characterized in that, in formula (I)
the ring B of the formula 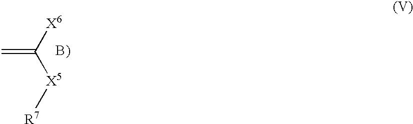
represents benzothiazol-2-ylidene, 1,3-thiazol-2-ylidene, isothiazol-3-ylidene, benzoxazol-2-ylidene, 1,3-oxazol-2-yliden isoxazol-3-ylidene, benzimidazol-2-ylidene, imidazol-2-ylidene, pyrazol-5-ylidene, 1,3,4-thiadiazol-2-ylidene, 1,3,4-oxadiazol-2-ylidene, 1,2,4thiadiazol-5-ylidene, 1,3,4-triazol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-ylidene or pyrrolin-2-ylidene, where X⁵ represents N and is substituted by R⁷, or the ring B represents 1,3-dithiol-2-ylidene, where X⁵—R⁷ represents S,
where the rings mentioned may each be substituted by C₁-C₆-alkyl, C₁-C₆-alkoxy, fluorine, chlorine, bromine, iodine, cyano, nitro, C₁-C₆-alkoxy-carbonyl, C₁-C₆-alkylthio, C₁-C₆-acylamino, C₆-C₁₀-aryl, C₆-C₁₀-aryloxy, C₆-C₁₀-arylcarbonylamino, mono- or di-C₁-C₆-alkylamino, N—C₁-C₆-alkyl-N—C₆-C₁₀-arylamino, pyrrolidino, morpholino or piperidino.

9. Optical data carrier as claimed in one or more of claims 1, 7 and 8, characterized in that the cationic aminoheterocyclic dye used is one of the formula (VI) 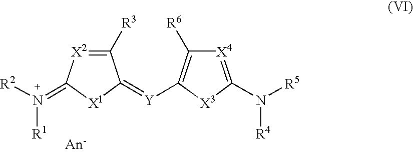
where
X¹, X³ and X³* represent, independently of one another, O, S or Se,
X² represents CR¹⁰ or N,
X⁴ and X⁴* represent, independently of one another, CR¹¹ or N,
R³ represents hydrogen, methyl, ethyl, propyl, butyl, chlorine, phenyl, tolyl, methoxyphenyl, xylyl or NR⁸R⁹,
R⁶ and R⁶* represent, independently of one another, hydrogen, methyl, ethyl, propyl, butyl, chlorine, phenyl, tolyl, methoxyphenyl, xylyl or NR¹²R¹³ or
R³ and R⁶ together form an —O—, —CH₂— or —C(CH₃)₂— bridge when n=0,
R¹, R², R⁴, R⁴*, R⁵, R⁵*, R⁸, R⁹, R¹², R¹³, R¹⁴* and R¹⁵* represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R², NR⁴R⁵, NR⁴*R⁵*, NR⁸R⁹, NR¹²R¹³ and NR¹⁴*R¹⁵* represent, independently of one another, pyrrolidino, morpholino, piperazino, N-methyl-piperazino or piperidino,
R¹⁰ and R¹¹ represent, independently of one another, hydrogen, cyano, methyl, ethyl, propyl, butyl, phenyl, tolyl, methoxyphenyl or xylyl,
Y represents ═Y¹—(Y²═Y³)ₙ— or N,
Y¹ to Y³ represent CH and Y¹ may also represent C—R¹⁸ when n=0,
R¹⁸ represents a radical of the formula 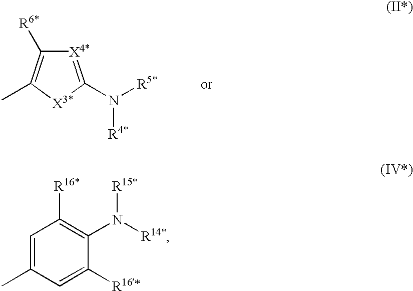
R¹⁶* and R^{16′}* represent, independently of one another, hydrogen, methyl, methoxy or chlorine or
R¹⁶*; R¹⁴* and/or R^{16′}*/R¹⁵* may form a —(CH₂)₂— or —(CH₂)₃— bridge,
n represents 0 or 1 and
An⁻ represents an anion.

10. Optical data carrier according to one or more of claims 1, 7 and 8, characterized in that the cationic aminoheterocyclic dye used is one of the formula (VII), 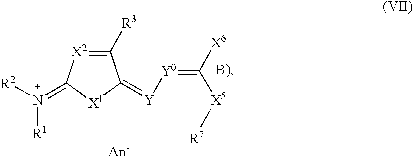
where
X¹ and X³* represent, independently of one another, O, S or Se,
X² represents CR¹⁰ or N,
X⁴* represents CR¹¹ N,
R³ represents hydrogen, methyl, ethyl, propyl, butyl, chlorine, phenyl, tolyl, methoxyphenyl, xylyl or NR⁸R⁹,
R⁶* represents hydrogen, methyl, ethyl, propyl, butyl, chlorine, phenyl, tolyl, methoxyphenyl, xylyl or NR¹²R¹³,
R¹, R², R⁴*, R⁵*, R⁸, R⁹, R¹⁴* and R¹⁵* represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R², NR⁴*R⁵*, NR⁸R⁹ and R¹⁴*R¹⁵* represent, independently of one another, pyrrolidino, morpholino, piperazino, N-methyl-piperazino or piperidino,
R₁₀ and R¹¹ represent, independently of one another, hydrogen, cyano, methyl, ethyl, propyl, butyl, phenyl, tolyl, methoxyphenyl or xylyl,
the ring B together with X⁵, X⁶ and R⁷ represents, when X⁵ represents N, benzothiazol-2-ylidene, 1,3-thiazol-2-ylidene, benzoxazol-2-ylidene, benzimidazol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-ylidene or pyrrolin-2-ylidene, where the heterocyclic radicals mentioned are each substituted on X⁵═N by R⁷, or, when X⁵—R⁷ represents S, represents 1,3-dithiol-2-ylidene, where the rings mentioned may each be substituted by up to three radicals selected from the group consisting of methyl, ethyl, trifluoromethyl, methoxy, ethoxy, fluorine, chlorine, bromine, cyano, nitro, methoxycarbonyl, ethoxycarbonyl, methylthio, formylamino, acetylamino, propionylamino, phenyl, tolyl, methoxyphenyl, phenoxy, benzoylamino, dimethylamino, diethylamino, dipropylamino, N-methyl-N-phenylamino, pyrrolidino, morpholino or piperidino,
R⁷ represents methyl, ethyl, propyl, butyl, cyanoethyl, hydroxyethyl, hydroxypropyl, allyl, cyclopentyl, cyclohexyl, benzyl, phenethyl or phenylpropyl,
Y represents ═Y¹—(Y²═Y³)ₙ— or N,
y¹ to Y³ represent CH and Y¹ may also represent C—R¹⁸, when n=0,
R¹⁸ represents a radical of the formula 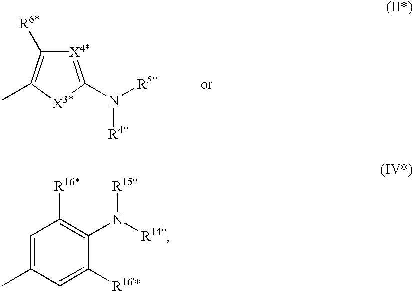
R¹⁶* and R^{16′}* represent, independently of one another, hydrogen, methyl, methoxy or chlorine or
R¹⁶*; R¹⁴* and/or R^{16′}*/ R¹⁵* may form a —(CH₂)₂— or —(CH₂)₃— bridge,
n represents 0 or 1,
Y⁰ represents CH or N and
An⁻ represents an anion.

11. Optical data carrier according to one or more of claims 1, 7 and 8, characterized in that the cationic aminoheterocyclic dye used has the formula (VIII), 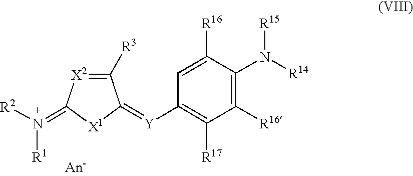
where
X¹ represents O, S or Se,
X² represents CR¹⁰ or N,
R³ represents hydrogen, methyl, ethyl, propyl, butyl, chlorine, phenyl, tolyl, methoxyphenyl, xylyl or NR⁸R⁹,
R¹, R², R⁸, R⁹, R¹⁴* and R¹⁵* represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R², NR⁸R⁹ and NR¹⁴*R¹⁵* represent, independently of one another, pyrrolidino, morpholino, piperazino, N-methyl-piperazino or piperidino,
R¹⁰ represents hydrogen, cyano, methyl, ethyl, propyl, butyl, phenyl, tolyl, methoxyphenyl or xylyl,
R¹⁴ and R¹⁵ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl, phenylpropyl, pyridyl or a radical of the formula 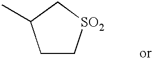
NR¹⁴R¹⁵ represents pyrrolidino, morpholino, piperazino, N-methyl-piperazino or piperidino,
R¹⁶ and R^{16′} represent, independently of one another, hydrogen, methyl, ethyl, methoxy, ethoxy or chlorine or
R¹⁶; R¹⁵ and/or R^{16′}; R¹⁴ form a —(CH₂)₂— or —(CH₂)₃— bridge which may be substituted by up to 3 methyl groups,
R¹⁷ represents hydrogen, methyl, ethyl, methoxy, ethoxy, chlorine, cyano, methoxycarbonyl, ethoxycarbonyl, O—CO—R²², NR²³—CO—R²², O—SO₂—R²² or NR²³—SO₂—R²²,
R²² represents methyl, ethyl, trifluoromethyl, cyclopentyl, cyclohexyl, benzyl, methoxy, ethoxy, dimethylamino, diethylamino, methylamino, phenyl, phenoxy, anilino or pyridyl,
R²³ represents hydrogen or methyl,
Y represents ═Y¹—(Y²═Y³)ₙ— or N,
Y¹ to Y³ represent CH and Y¹ may also represent C—R¹⁸ when n=0,
R¹⁸ represents a radical of the formula 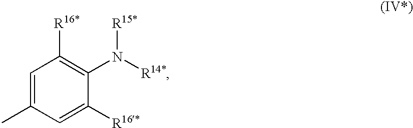
R¹⁶* and R^{16′}* represent, independently of one another, hydrogen, methyl, methoxy or chlorine or
R¹⁶*; R¹⁴* and/or R^{16′}*/R¹⁵* may form a —(CH₂)₂— or —(CH₂)₃— bridge,
n represents 0 or 1 and
An⁻ represents an anion.

12. Cationic aminoheterocyclic dyes of the formula (VI), 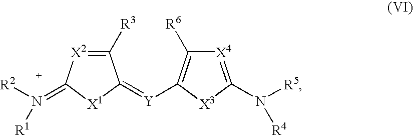
where
X¹ and X³ represent, independently of one another, O or S,
X² represents CR¹⁰ or N.
X⁴ represents CR¹¹ or N,
R³ represents hydrogen, methyl, ethyl, propyl, butyl, chlorine, phenyl, tolyl, methoxyphenyl, xylyl or NR⁸R⁹,
R⁶ represents hydrogen, methyl, ethyl, propyl, butyl, chlorine, phenyl, tolyl, methoxyphenyl, xylyl or NR¹²R¹³ or
R³ and R⁶ together form an —O—, —CH₂— or —C(CH₃)₂— bridge when n=0,
R¹, R², R⁴, R⁵, R⁸, R⁹, R¹² and R¹³ represent, independently of one another, hydrogen, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R², NR⁴R⁵, NR⁸R⁹ and NR¹²R¹³ represent, independently of one another, pyrrolidino, piperazino, N-methyl-piperazino or piperidino,
R¹⁰ and R¹¹ represent, independently of one another, hydrogen, cyano, methyl, ethyl, propyl, butyl, phenyl, tolyl, methoxyphenyl or xylyl,
Y represents ═Y¹—(Y²═Y³)ₙ— or N,
Y¹ to Y³ represent CH,
n represents 0 or 1 and
An⁻ represents an anion.

13. Cationic aminoheterocyclic dyes of the formula (VII), 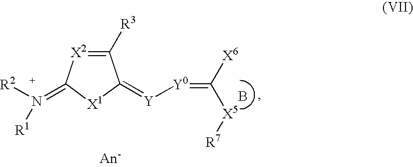
where
X¹ represents O or S,
X² represents CR¹⁰ or N,
R³ represents hydrogen, methyl, ethyl, propyl, butyl, chlorine, phenyl, tolyl, methoxyphenyl, xylyl or NR⁸R⁹,
R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R² and NR⁸R⁹ represent, independently of one another, pyrrolidino, morpholino, piperazino, N-methyl-piperazino or piperidino,
R¹⁰ represents hydrogen, cyano, methyl, ethyl, propyl, butyl, phenyl, tolyl, methoxyphenyl or xylyl,
the ring B together with X⁵ and X⁶ represents, when X⁵ represents N, benzothiazol-2-ylidene, 1,3-thiazol-2-ylidene, benzoxazol-2-ylidene, benzimidazol-2-ylidene, 3-H-indol-2-ylidene, dihydropyridin-2- or -4-ylidene, dihydroquinolin-2- or -4-ylidene, 1,3-thiazolin-2-ylidene, 1,3-oxazolin-2-ylidene, imidazolin-2-ylidene or pyrrolin-2-ylidene or, when X⁵-R⁷ represents S, represents 1,3-dithiol-2-ylidene, where the rings mentioned may each be substituted by up to three radicals selected from the group consisting of methyl, ethyl, trifluoromethyl, methoxy, ethoxy, fluorine, chlorine, bromine, cyano, nitro, methoxy-carbonyl, ethoxycarbonyl, methylthio, formylamino, acetylamino, propionylamino, phenyl, tolyl, methoxyphenyl, phenoxy, benzoyl-amino, dimethylarnino, diethylamino, dipropylamino, N-methyl-N-phenylamino, pyrrolidino, morpholino or piperidino,
R⁷ represents methyl, ethyl, propyl, butyl, cyanoethyl, hydroxyethyl, hydroxypropyl, allyl, cyclopentyl, cyclohexyl, benzyl, phenethyl or phenylpropyl,
Y represents ═Y¹—(Y²═Y³)ₙ— or N,
Y¹ to Y³ represent CH,
n represents 0 or 1,
Y⁰ represents CH or N and
An⁻ represents an anion.

14. Cationic aminoheterocyclic dyes of the formula (VIII), 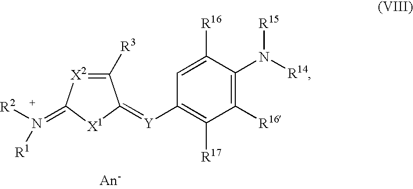
where
X¹ represents O or S,
X² represents CR¹⁰ or N,
R³ represents hydrogen, methyl, ethyl, propyl, butyl, chlorine, phenyl, tolyl, methoxyphenyl, xylyl or NR⁸R⁹,
R¹, R², R⁸ and R⁹ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl or phenylpropyl or
NR¹R² and NR⁸R⁹ represent, independently of one another, pyrrolidino, piperazino, N-methyl-piperazino or piperidino,
R¹⁰ represents hydrogen, cyano, methyl, ethyl, propyl, butyl, phenyl, tolyl, 20 methoxyphenyl or xylyl,
R¹⁴ and R¹⁵ represent, independently of one another, hydrogen, methyl, ethyl, propyl, butyl, cyanoethyl, hydroxyethyl, hydroxypropyl, cyclopentyl, cyclohexyl, phenyl, tolyl, methoxyphenyl, xylyl, benzyl, phenethyl, phenylpropyl, pyridyl or a radical of the formula 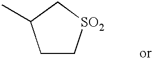
NR¹⁴R¹⁵ represents pyrrolidino, morpholino, piperazino, N-methyl-piperazino or piperidino,
R¹⁶and R^{16′} represent, independently of one another, hydrogen, methyl, ethyl, methoxy, ethoxy or chlorine or
R¹⁶; R¹⁵ and/or R^{16′}; R¹⁴ form a —(CH₂)₂— or —(CH₂)₃— bridge which may be substituted by up to 3 methyl groups,
R¹⁷ represents hydrogen, methyl, ethyl, methoxy, ethoxy, chlorine, cyano, methoxycarbonyl, ethoxycarbonyl, O—CO—R²², NR²³—CO—R²², O—SO₂—R²² or NR²³—SO₂—R²²,
R²² represents methyl, ethyl, trifluoromethyl, cyclopentyl, cyclohexyl, benzyl, methoxy, ethoxy, dimethylamino, diethylamino, methylamino, phenyl, phenoxy, anilino or pyridyl,
R²³ represents hydrogen or methyl,
Y represents ═Y¹—(Y²═Y³)ₙ— or N,
y¹ to Y³ represent CH,
n represents 0 or 1 and
An⁻ represents an anion.

15. Use of cationic aminoheterocyclic dyes in the information layer of write-once optical data carriers, where the cationic aminoheterocyclic dyes have an absorption maximum λₘₐₓ₂ in the range from 420 to 650 nm or a λₘₐₓ₃ in the range from 650 to 810 nm.

16. Use of cationic aminoheterocyclic dyes in the information layer of write-once optical data carriers, where the data carriers can be written on and read by means of blue, red or infrared laser light.

17. Process for producing the optical data carriers according to claim 1, which is characterized in that a preferably transparent substrate which may, if desired, have previously been coated with a reflection layer is coated with the cationic aminoheterocyclic dyes, if desired in combination with suitable binders and additives and, if desired, suitable solvents, and is, if desired, provided with a reflection layer, further intermediate layers and, if desired, a protective layer or a further substrate or a covering layer.

18. Optical data carriers according to claim 1 which have been written on by means of blue, red, infrared, in particular red light, in particular red laser light.
